(19) United States
(12) Patent Application Publication
(10) Pub. No.: US 2021/0267985 A1
(43) Pub. Date: Sep. 2, 2021

(54) COMPOSITIONS AND METHODS FOR INDUCING ENHANCED BRAIN FUNCTION

(71) Applicant: Gerald Horn, Deerfield, IL (US)

(72) Inventor: Gerald Horn, Deerfield, IL (US)

(21) Appl. No.: 17/187,688

(22) Filed: Feb. 26, 2021

Related U.S. Application Data
(60) Provisional application No. 62/982,169, filed on Feb. 27, 2020.

(51) Int. Cl. A61K 31/522; A61K 36/82; A61K 36/06; A61K 33/00; A61K 9/00; A61K 31/7004; A61K 31/198

(52) U.S. Cl. CPC A61K 31/522; A61K 36/82; A61K 36/06; A61K 31/198; A61K 9/0095; A61K 31/7004; A61K 33/00

(57) ABSTRACT

The present invention is directed to compositions and methods to induce enhanced brain function and brain and systemic recovery.

### FIELD OF THE INVENTION

The present invention is directed to compositions and methods to induce enhanced brain function and brain and systemic recovery.

### BACKGROUND OF THE INVENTION

A considerable challenge to optimizing brain health and providing sustainable long duration mental focus and concentration or related cognitive effort, is evolutionary adaptive sensitivity of brain biochemical responsiveness, particularly metabolism, to even slight fluxes in glucose levels. Not wishing to be held to particular theory, small transient drops in cerebral serum glucose, such as following consumption of a zero calorie beverage and more particularly a caffeinated energy drink or black coffee without stable sustained adequate glucose levels, may result in metabolic conversion to lactose by brain cells over glucose as a fuel source despite its marked inefficiency for that purpose. Spikes in serum glucose levels on the other hand, as triggered by ingestion of high glycemic sugar loads induce exaggerated or at least proportionate increase in insulin release, and insulin resistance may occur, particularly when metabolic demands are high and temporarily or eventually permanently reduce glucose cell permeability.

Even in the absence of systemic insulin resistance/diabetes a form of “cerebral diabetes” may occur, where insulin resistance may not exist systemically but does cerebrally. High glycemic index sugar loads, such as a high fructose corn syrup or other high glycemic carbohydrate laced energy drink may adversely compromise brain metabolism, response to brain stimulants such as caffeine, and produce toxic cytokines, induce neuron excitotoxicity and create local brain and systemic side effects reducing safety and efficacy of caffeine or other brain stimulants. Similarly, intake of fatty foods may spike cerebral serum lipopolysaccharide (lps) levels, and in addition other nutritional toxicity, such as ingestion of heavy metals and or other pollutants or toxins, may exacerbate brain inflammation and further contribute to functional degradation of brain function, spreading a gradient of functional impairment via inflammatory interleukins, tumor necrosis factors and or other cytokines that further compromise use of any brain stimulant such as caffeine.

The absorption and delivery across the blood brain barrier to the cerebral circulation of “suboptimal” brain fuel dominates the limited nutritional options of our processed food, high glycemic load sugar cravings and high saturated fat diets, and that increasingly typify “modern” society. It is exacerbated frequently by ingesting high glycemic index carbohydrates such as processed food with high levels of for example fructose corn syrup, leading to fast absorption and conversion to glucose, triggering excessively high postprandial insulin release with risk of reduced glucose permeability by brain cells secondary to reduced insulin sensitivity and or exhaustion. This pattern within the cerebral circulation, though mirroring that found in peripheral circulation in diabetics, may particularly occur even when systemic insulin function and peripheral glucose serum levels and response to interested glycemic loads is normal.

Artificially sweetened zero-calorie or low-calorie drinks typical of modern attempts at weight control may contribute to sudden drops in cerebral circulation glucose levels. The exquisite sensitivity of brain cells to flux in cerebral serum glucose levels, which mirrors to a great extent peripheral levels, is therefore easily compromised following intake of food and or beverages. Disturbances in the flora of the gut biome based on quality of our general nutrition as well as more acutely any anti-infective medications (e.g. antibiotics) taken can further limit attempts to induce and optimize brain metabolism.

An emerging body of evidence suggests that mental processing and capacity is not only temporally enhanced by brain stimulants such as caffeine, but brain stimulants such as caffeine may possibly offer a degree of protection from cognitive decline, such as in generalized forms of dementia, Alzheimer's or LATE, (limbic-predominant age-related TDP-43 encephalopathy), an Alzheimer's-like condition common above age 80.

Caffeine is a stimulant-primarily an adenosine receptor antagonist, reducing the desire to sleep and thereby extends the range of “wakefulness” and time brain function at higher levels of metabolism is on demand with prolonged periods without rest. The effects of caffeine are complex. Though generally considered a stimulant that is typically true initially for short periods but rapidly degraded by rapidity of ingestion increasing serum concentration spikes and reducing total caffeine versus tine of ingestion typical of cold caffeinated beverages.

Caffeine toxicity and overstimulation creates neuronal excitotoxicity, and can lead to neuronal, synapse cerebral metabolic “burn out”, producing lethargy, mental and physical fatigue, even prolonged insomnia exacerbated by high quickly absorbed caffeine bolus doses typical of caffeinated beverages; even states of transient psychosis with high caffeine bolus that could be attributed to brain cell induced toxicity gradients.

Reasons energy drinks deliver high onset doses of caffeine ranges from the “jolt systemic charge” to some desire, to bring the byproduct of high caffeine level early onset spikes of bolus dumps of caffeine. Such as when anhydrous caffeine powder is added to water. Another reason is because there is limited means in the absence of methods and formulations to calibrate caffeine dose versus time in a beverage to achieve longer duration of alertness effect or other cognitive benefit or protection while simultaneously controlling desired higher or more gradual onset. For example for sports performance, physical workouts, gaming, or other activities where higher quicker onset brain stimulation and energy are desired or needed, versus work, study related, or high skill performance related tasks (e.g. nurse, pilot, physician, surgeon, programmer) or any task where long duration intense concentration is needed or where emphasis is in high quality longer duration brain energy/alertness but early fast onset is not desired additionally.

Despite a 4-6 hour half-life of caffeine, without optimized brain metabolism caffeine levels more quickly reduce efficacy and become marginal and or sub-therapeutic; so the side effect toxicity profile is considerably higher yet the need to enhance caffeine's benefits is as well, creating a potential cycle of caffeine abuse, limited duration efficacy, and greater induction of toxic side effects. Energy drink toxic sensitivity or abuse has been a cause of increased emergency room visits and even rarely deaths.

High glycemic sugar levels often present may further exacerbate glucose control by diabetics including induced keto-acidosis; yet artificial sweeteners otherwise used contributes potential toxicity with no contribution to effective brain metabolism in the face of induced brain stimulation. Studies in healthy male volunteers have demonstrated abnormal EKG change (e.g. prolonged QT intervals) even in the absence of other demonstrable side effects.

What is needed is a formulation and method for overcoming these baseline limitations to brain stimulation following caffeine or other brain stimulants for the purpose of enhanced wakefulness, concentration, focus, and enhanced cognition; with these stimulant effects preferably juxtaposed with a of a state of optimized metabolism, no to reduced increased oxidative stress, no to reduced induction of inflammation. All are only minimally achieved, substantially less effective relative to the present invention and or largely absent from prior art coffees, teas, energy drinks, and nootropic supplements.

Thus, there is a need in the art for a composition that provides brain enhancement that is long lasting and does not result in a subsequent deficit in brain function and discovers compositions and means to deliver the full spectrum of benefits of said brain stimulants. The cure or prophylactic prevention of cognitive decline, dementia, Alzheimer's disease, or similarly LATE in the elderly is also needed.

### SUMMARY OF THE INVENTION

The present invention is directed to compositions to induce enhanced brain function and brain and systemic recovery comprising:

a. a brain stimulant derived from a source selected from the group consisting of caffeine powder, cold or hot brewed caffeinated roast coffee, a non-caffeine stimulant and a combination thereof;
b. one or more beverages selected from the group consisting of caffeine powder enhanced with L-theanine, unroasted green coffee bean extract, black tea, white tea, green tea, green tea concentrate, and matcha tea; and
c. an energy source selected from the group consisting of a low glycemic carbohydrate, preferably the low glycemic carbohydrate is selected from the group consisting of D-ribose, fructose, lactose and sucrose, a medium chain saturated triglyceride, preferably selected from the group consisting of C8, C10, and C12 fatty acids, a source of amino acids, preferably the source of amino acids is selected from the group consisting of protein, peptides, collagen, hydrolyzed collagen, and twice hydrolyzed collagen.
The present invention is further directed to methods of enhancing brain function and brain and systemic recovery comprising administering to a subject in need thereof a composition of the present invention.

### BRIEF DESCRIPTION OF THE DRAWINGS

FIG. 1. The addition of zinc at 250, 500, and 750 mg to Breinfuel™ resulted in increased antiviral and antibacterial titers.

FIG. 2. The addition of GCBE and green tea extract to Breinfuel™ resulted in decreased levels of plasma oxidative stress markers.

### DETAILED DESCRIPTION OF THE INVENTION

The ability to optimize brain metabolism, and safely enhance brain activity following metabolic induction by a brain stimulant, is critical to optimal brain stimulation from any source, including but not limited to coffee, guarana, carnitine, conjugated linoleic acid, forskolin, chromium picolinate, fucoxanthin, ephedrine, THC, hallucinogens, amphetamines or amphetamine derivative medications such as Adderall® (amphetamine & dextroamphetamine). It is conditional on and particularly adversely sensitive to any flux or other aberrations in glucose stability, fatty food influx, or other nutritional toxicity, deprivation or deviation such as via liquid cleanses or single food diets or fads.

The suboptimal and frequently inferior availability of adequate antioxidant levels and or fuel for brain metabolism in the presence of brain stimulant(s) may cause oxidative stress, inflammation, and related toxicity or excitatory neurotoxicity artificially degrading, limiting, or even destroying these potential inductive and or protective benefits to neuronal brain health, at a minimum confounding optimal alertness, and cognitive performance, with such stimulant use.

Achieving optimal brain function is therefore an elusive rarely achieved state in prior art nootropics as well as prescription medications not addressing these transient and sustained predominant brain metabolic deficiencies. requiring a random virtually impossible highly synchronized combinations moment to moment timing of one or more of stable glucose levels, maintaining low lps levels, low glycemic loads, alternative early metabolized non-glucose brain fuels superior to lactose, ancillary antioxidants, amino acid neurotransmitter precursors; al in the presence of absent or low levels of inflammatory changes in brain cells or their surrounding milieu. To induce a prolonged state of superior mental alertness, and it affect a reduced rates of cognitive decline with age or disease process, and or partially improve cognitive performance and or ameliorate diseases causing dementia; all of the above requires expanding these moment to moment requirements to be more controllable synchronized over many hours in the presence of brain stimulants and even in their absence.

Brain stimulants, and necessary corollary increased efficient brain metabolism can be optimal only if simultaneous stabilization of glucose levels occurs, and preferably alternative non-glucose oxidative metabolic fuels are available. It is a discovery of the present invention that combinations of caffeine or other brain stimulants, including but not limited to roasted coffee, with green coffee beans, their extracts, or one or more teas, for the duration of such stimulation results in prolonged enhanced brain function with greatly reduced or absent side effects and may induce proportionally increased endorphin release, particularly in the presence of low glycemic sugars, medium chain fatty acids, ketones, proteins and or amino acids, and that such combinations offer profoundly enhanced brain function.

Deleterious oxidative stress is a byproduct of impaired metabolism in conjunction with metabolic stimulant triggers, particularly in the absence of stable sustainable glucose levels or any toxicity or inflammation, and metabolism in the presence of brain stimulation may be further optimized by addition of one or more antioxidants.

Lithium, and particularly micro doses of lithium may induce inotropic interleukins or other anti-inflammatory precursors (such as demonstrated via IL-2, IL-10, class of 17-OH-DHA omega3's) and reduce, prevent, enhance recovery from, or reverse tissue damage from stimulant induced metabolic stress and inflammation.

Mushroom extract(s), including particularly lions mane, and or one or more of reishi, enotake, maitake, chaga, royal blazeii and or cordyceps may contribute one or more of anti-oxidant, anti-inflammatory, neuroprotective, neurite stimulating, and or other benefits to synapses, myelination, neuronal health and recovery time, and overall brain health; including through inotropic factors they contain such as polyphenols, polysaccharides, proteases, terpenoids, lectins, illudins, ribosome inactivating proteins, vitamins and minerals contained therein. Such extracts thereby may facilitate better brain stimulant survival, recovery and potentiation.

The quality of our nutrition on a per ingestion basis serves to potentially affect brain metabolic processes on an almost direct and near immediate basis, limiting brain metabolic added capacity for any accelerated metabolic demand, or even baseline function, may produce a transient “brain fog” that added brain stimulants may worsen. As a result, prior to the present invention, most humans have rarely if ever experienced optimal brain metabolism for a sustained period of time, and particularly not after use of caffeine, amphetamine derivatives, or other brain stimulants. Adaptive mechanisms of the human body to or nutritional intake that reduce optimal glucose metabolism, and as a result optimal alertness must be in some beneficial way controlled versus prior art for optimized brain stimulation.

Initial contributions to mental alertness by caffeinated beverages may only achieve partial effect, and less likely to include or with regular use be followed by any or full potential for improved cognitive function, if toxic elements of high dose spikes and transient and even high levels of sustained toxicity are a confounding component. At high doses caffeine toxicity may predominate degrading positive stimulant affects.

Most energy drinks for example induce quick onset sudden spikes in caffeine peripheral and cerebral serum levels for part of their alertness; as a “jolt” to the brain to create an exhilarating initial rush, later often followed by a mental “crash”. Rating beverages or complements with caffeine based on the total caffeine may be appropriate for prior art as repopulated to rush off side effects. However forties beverages, such as the present invention, may discover ways to optimize one or of 1) rates of caffeine absorption versus time; 2) tastes if adaptability glycemic optimized fuels; 3) rates of antioxidant delivery to reduce high metabolism oxidative stress; and provide ant-inflammatory immune-modulation making the total caffeine dose no longer as predictive or even predictive at all in ranges previously thought to be high or toxic. In fact, such pressure art designated high or toxic caffeine ranges may be more potent less toxic in the presence of such inventions than possible with prior art, abs may even lead to discoveries they can reduce and or reverse cognitive decline.

The previous paragraphs are based in theory and the Applicant does not wish to be held to any particular theory as it relates to the previous paragraphs.

The present invention targets separate modulation of low to high early release with greater safety; extends caffeine effective duration; synchronizes key metabolic fuels versus time of caffeine induced extended release; control of timed release from a variety of caffeine sources beyond their norm discovered to be further enhanced by the type and excipients in the metabolic fuel blend; reduction of oxidative stress and inflammation; and additional unique health benefits. Juxtaposing molecular caffeine with discoveries of enhanced quick or gradual release as well as long duration or other brain stimulant such as Adderall®, with one or more of micro dose lithium, vitamin C or other antioxidant(s), and a blend of mushroom extracts such as lions mane, and one or more of chaga, reishi, cordyceps, promotes discovery of a more complete positive physiologic enhanced benefit versus toxicity profile.

Our current struggle to identify a single drug that can effectively prevent dementia, or at least reduce cognitive decline, or possibly treat said afflictions, may instead depend on the combination of optimized brain stimulation via caffeine or other brain stimulants with the additional additives of the present invention and their regular use.

In one embodiment, the present invention is directed to compositions to induce enhanced brain function and brain and systemic recovery comprising:
a. a brain stimulant derived from a source selected from the group consisting of caffeine powder, optionally combined in a ratio with L-theanine, cold or hot brewed caffeinated roast coffee, a non-caffeine stimulant and a combination thereof;
b. one or more beverages selected from the group consisting of unroasted green coffee bean extract, black tea, white tea, green tea, green tea concentrate, and matcha tea; and
c. an energy source selected from the group consisting of a low glycemic carbohydrate, preferably the low glycemic carbohydrate is selected from the group consisting of D-ribose, fructose, lactose and sucrose, a medium chain saturated triglyceride, preferably selected from the group consisting of C8, C10, and C12 fatty acids, a source of amino acids, preferably the source of amino acids is selected from the group consisting of protein, peptides, collagen, hydrolyzed collagen, and twice hydrolyzed collagen.

In a preferred embodiment, the compositions of the present invention comprise lithium, more preferably lithium is provided in compositions of the present invention in a dose that is less than 1 mg.

In another preferred embodiment, the compositions of the present invention comprise one or more additional antioxidant(s), more preferably the antioxidant is particularly vitamin C.

In another preferred embodiment, the compositions of the present invention comprise one or more mushroom extract(s), more preferably the mushroom extracts are selected from the group consisting of lions mane, reishi, enotake, chaga, maitake, and cordyceps, and even more preferably lions mane is in an alcohol free extract.

The present invention further discovers a ratio of from 100 to 120 mg caffeine anhydrous to 160 to 200 mg L theanine, in addition to other coffee or tea caffeine sources, produces preferred optimal early onset high intensity alertness.

The present invention further discovers a ratio of from 80 mg caffeine anhydrous to 120 to 140 mg L theanine, in addition to other coffee or tea caffeine sources, produces preferred seamless onset alertness and focus.

The present invention may further comprise additional ingredients selected from the group consisting of citric acid, creatine, beet root powder, potassium (K) sorbate, sodium (Na) benzoate, vitamin E acetate 50% CWS/S and zinc including zinc salts such as zinc citrate and hydrates thereof such as zinc citrate dihydrate.

Formulations disclosing preferred embodiments for stress-relief/panic attacks; relaxation with alertness; sleep promotion; cognitive enhancement; Alzheimer's prevention; immune boost; cancer prevention; suicide ideation suppression are disclosed herein.

Compositions having the combination of a.-c. from paragraph [028] above, and even more effectively with one or more of the additional ingredients from paragraphs [029] to [031] result in novel enhanced brain function, including:
i) smoother, more gradual onset of enhanced alertness at up to about 400-500 mg of caffeine per 8-16 oz serving;
ii) more prolonged periods of sustained and intensified mental focus;
iii) controlled delay of gastric emptying and caffeine or other brain stimulant (intestinal) absorption;
iv) greatly reduced or absent side effects including those of nervousness, shakes, jitteriness, tremors, anxiety, restlessness, insomnia, elevated heart rate or clinically significant blood pressure elevation, urinary frequency, sudden mental fatigue, mental “crash”, or transient inability to trigger intentioned thought formation (“brain fog”);
v) a state of simultaneous alertness and anxiolysis, calmness and enhanced power of concentration, in which a tranquil state is juxtaposed with an acutely increased and prolonged mentally sharp focused one;
vi) ease of sleep transition, where the effort to sleep induces enhanced somnolence and deeper quality sleep, accelerated recovery from fatigue or sleep deprivation, pain relief—particularly on arising up to 12/24 hours later and feelings associated with positive endorphin release such as an overall greater sense of rest, well-being, and or clarity of thought, and where this affect may be greatest 12/24 hours or more post ingestion;
vii) faster transmitter replenishment with essential amino acid delivery and branched chain amino acid delivery in particular;
viii) the option to selectively modify onset time, onset level, duration to effectively customize varied desired performance curves of caffeine versus time and metabolic synchronizing optimal fuels versus time;
ix) the release of anxiolytics found in various caffeine sources and amino acids;
x) resulting in varied range of therapeutic use from brain energy with musculoskeletal stimulation, brain energy alone, brain energy with a sense of relaxation, anxiolysis, reduction of panic attacks, endorphin promotion, faster sleep onset, quality, and recovery;
xi) it is prophetically predicted that with regular use the present invention can enhance cognitive function, reduce the rate of cognitive decline and or reverse its degree; and
xii) it is prophetically predicted that alternative brain receptor transmitters, such as those replacing or reducing serotonin receptor uptake, and or hallucinogens, such as Ayahuasca, DMT, MDMA and or psilocin/psilocybin extracts or mushroom exposure may be positively enhanced in the presence of the present invention particularly when micro-doses below level of hallucinogens; and may more effectively potentiate a positive experience, and further potentiate multiple medical benefits, including but not limited to psychosocial, anti-depressive, panic or post-traumatic stress related, general anxiolytic, and with frequent or daily use facilitate cognitive enhancement, greater amelioration of one or more dementias; and may facilitate neuronal pathway recovery from cerebrovascular stroke, cranial nerve injury including hearing or vision related nerve deficits, other brain tissue degeneration or malformation, generalized brain disfunction including cerebral palsy, autism, bipolar disorders, personality disorders, criminal, deviant or violence prone behavioral disorders.

In another preferred embodiment, the compositions of the present invention may further comprise stimulants other than, inclusive of, or in addition to caffeine, such as guarana, forskolin, chromium picolinate, ephedrine, amphetamine, amphetamine and dextroamphetamine (Adderall®), hallucinogens or other metabolic, neuroprotective, or neurogenic stimulants.

In another embodiment, the present invention is directed to methods of enhancing brain function and brain and systemic recovery comprising administering to a subject in need thereof a composition of the present invention.

The present invention further discovers compositions and means to enhance caffeine benefits while reducing caffeine's systemic side effects. The invention discovers safer and more potent long-lasting compositions and means to enhance alertness, extend the duration of mental concentrated effort, reduce or eliminate subsequent mental fatigue, and enhance the ability to transition to restful sleep often with accelerated sleep recovery without harmful systemic side effects at caffeine concentrations as high as 400-500 mg up to twice a day. Optimizing mental effort, prolonging it during periods of consciousness, and typically accelerating sleep cycle recovery still requires total sleep time that “catches up” periodically, consistent with known individual daily requirements on any long-term basis.

Not wishing to be held to particular theory, just as an individual cannot run effectively if their shoes are too tight, or an athlete is adversely constrained if any source of poor circulation is present in an extremity, the resulting ischemia causes lack of or reduced oxidative metabolism of nutrients and fuel, conversion to non-oxygen metabolism via lactic acidosis, and inefficient fuel conversion to metabolic energy causing cramping, local toxic inflammation with release of interleukins and tumor necrosis factors that can substantially degrade musculoskeletal performance within tens of seconds and if continued eventually prevent its useful continuation. The brain, and brain metabolism is according to this theory similarly constrained when stimulated without adequate oxidative fuel sources for oxidative metabolism, and is similarly constrained under conditions of increased oxidative stress, delivers a near immediate conversion to low energy conversion lactic acid (lactose), becomes metabolically impaired, with degradation of brain function and eventually risk of increasing mental fatigue, if continued mental “crash” requiring immediate rest, and if further stressed with forced concentration or additional mental effort a “brain fog” where any intended thought or short term memory recall is transiently at least severely disrupted.

The combination of stimulant laced energy drinks with fuel deprivation or compromise, such as with zero calorie energy drinks, high glycemic loads, or high lipopolysaccharide spikes, according to this theory, often impairs systemic and cerebral metabolism, increases oxidative stress and increases local cerebral inflammation with induced brain fatigue and lethargy that perpetuates a dangerous cerebral and systemic caffeine/stimulant cycle, with toxicity ranging from induced hypertension and tachycardia, to vasoconstriction and cardiovascular compromise, including prolonged qt waves seen on E.K.G.'s and possibly greater risk of stroke and or infarction in addition to impaired cerebral function. When high quantities for example of caffeinated energy drinks are rapidly consumed, even with much lower doses of caffeine than in the present invention these side effects and possible emergent complications including risk of a fatal outcome may otherwise occur.

Drinking hot coffee or tea spreads out dosing to the ingest the full quantity of caffeine and adds protective antioxidants that together slightly ameliorated the toxicity of energy drinks. The time frame for consumption is not appears practical, and the inadequacy of brain fuels, oxidative stress, and inflammation if cumulative doses are significant limits these benefits. Several cups of coffee, green tea, or matcha green tea are required to get greater benefit if inferior prior art caffeine utilization. Unfortunately, caffeine toxicity increase, and some components of green tea can be hepatotoxic in high doses.

Prior caffeinated drinks, whether energy drinks, medium or dark roast coffee, green tea, or green matcha tea; green coffee bean extracts or other caffeinated nootropic supplements; all induce caffeine spikes and or brain stimulation frequently with added high glycemic toxicity and without optimized brain cell metabolism. Frequent use required may induce toxicity ranging from jitters and shakiness, to brain fog; and cardiovascular or other systemic toxicity including keto-acidotic coma or liver failure that are life threatening.

Nootropics, or “smart drugs,” are a class of substances that claim to boost brain performance. They are sometimes called cognition enhancers or memory enhancing substances, and frequently claim effectiveness demonstrating cognitive improvement or even evidence of benefit in treating dementia or Alzheimer's disease. Caffeine is occasionally added. Claims made regarding nootropic supplements inducing cognitive enhancement have been largely unsupportable by any clinical studies in humans. In 2019 the FDA cited 18 manufacturers for unproven or exaggerated claims about their use. No members of this class consisting of hundreds if not thousands of supplements, not any other prior art duplicate or predict the surprising effective discoveries of the present invention.

A prolonged, sustainable potentiation of the alertness of the human brain using novel compositions of caffeinated beverages, without the side effects or limited effectiveness for this purpose of prior art coffees, teas, energy drinks, beverages, or nootropic supplements, is discovered by the present invention.

The surprising and unexpected novel compositions and methods discover a ‘brain metabolism’ optimized, nutrient dense, low glycemic energy blend when added to a blend of coffees, green tea and green coffee bean extracted derivatives becomes a highly potent selective inducer of alertness and mental focus allowing hours of imperceptible smooth onset sustained concentration and feeling of well-being without any of the “jolt”, shakiness, jitters, insomnia, brain fog, energy crash, transient psychosis, induced hypertension, tachycardia, arrhythmias, sugar cravings, or keto-acidotic emergencies associated with prior art caffeinated drinks, beverages, nootropics and or energy drinks.

By combining a low to moderate caffeine liquid coffee base and or caffeine powder, with one or more of L-theanine caffeine ratio modulation, green coffee bean extract, matcha tea powder; and two or more of a nutrient dense blend of a low glycemic carbohydrate such as D-ribose, medium chain triglyceride ketones, and proteins, the present invention discovers new compositions and methods that induce and sustain high levels of mental alertness for long durations, induce a feeling of well-being, and are safer with repetitive use. In a preferred embodiment these benefits are further facilitated if the proteins include hydrolyzed collagen.

Not wishing to be held to particular theories, based on these discoveries it appears caffeinated drinks or supplements whether coffees and or teas, energy drinks, or nootropic supplements will provide an amplified benefit with greatly reduced to absent toxicity when a multi-source nutrient dense blend of low glycemic non-glucose carbohydrate sugar alternatives, ketones, proteins, and or amino acids is combined therein;

Not wishing to be held to particular theory it appears that one or more of an antioxidant(s), low dose or micro dose lithium, and mushroom extract(s) further contributes to brain function, alertness, and other health benefits for overall endorphin type benefits of reduced anxiety, greater tranquility and greater alertness with the present invention.

It is further held that caffeine brain stimulation without the discoveries of the present invention adds a gradient of destructive metabolic toxicity; which with repetitive use compounds such adverse gradients and results in substantial deleterious effects that confound and reduce its capacity to Enhance brain function; and that caffeine and particularly the caffeine blend of the present invention with sources of caffeine, optimized low glycemic fuels or one or more of the proteins, amino acids and select fatty acids allows more prolonged and efficient glucose stabilized brain function.

Not wishing to further be held to any particular theory, the high nutrient density of this fuel blend provides not only improved brain metabolism, but appetite suppression. A reduced craving of sweets or other high glycemic sugars, of high fatty high lipopolysaccharide meals, and better portion control typically follow its ingestion. It may be used as a meal replacement, to break an intermittent fast, or on a keto diet. A preferred embodiment includes a cold brewed 270 mg of caffeine, about 150 calories, 5 g D-ribose, 10.5 g collagen protein, 9 g peptides, 6 g medium chain triglycerides (C8/C10/C12 in about 7.5/5/0.7 ratio), about 7 g total fat as saturated fat, 0 trans-fat, 0 cholesterol, less than 15 g carbohydrates and 7 g as sugar. In addition to facilitating brain metabolism directly by inducing significant satiety, the appetite suppression that results by reducing high sugar and rich fatty food cravings may reduce substantial negative nutritional effects on brain metabolism which will otherwise degrade and slow brain metabolism and intended caffeine potential benefit, to which the caffeine and or other brain stimulated brain may be particularly sensitive; and that these indirect benefits of its nutrient density onbdatiety, in addition to benefitting brain metabolism directly add a key, surprising and necessary added benefit for optimal brain stimulation effectiveness.

Preferred embodiments may add low or micro-doses of lithium, not wishing to be held to theory may contribute to the inventions surprising effectiveness as well. Lithium at high doses of typically 1200 mg (1.2 M mcg)/day treats bipolar disorder. Municipal water supplies often contain lithium particularly near aquifers. A peer reviewed Danish study using national health care statistics showed positive inverse correlations with dementia and suicide at or above 15 mcg/l. Similar data was found in a study regarding Texas municipalities. (A published letter-not peer reviewed) adjusting for confounding data in the Texas study using 40 mcg/1 or higher as the high lithium water supply definition found less of no significance). In USGS Surveys of 3557 counties measured 26% were at or above 0.15 mcg with a mean of 60 mcg/l, of which 14.4% are between 0.15 and 0.40 mcg/l. The high was 1234.1 mcg/1 (Carroll County, NH), with 17 counties over 250 mcg/1, and 36 above 200 mcg/l. Lithium orotate is a mineral commonly available as a supplement not regulated by the FDA as lithium orotate.

Not wishing to be held to particular theory, high doses used for clinical treatments versus micro doses of the present invention may be less useful or protective for increased brain metabolism as they are for bipolar and related disorder, and cognitive benefits may be promoted more effectively at micro doses. Higher dose often 900 mg per day or greater with prescription-based use of lithium carbonate as a salt may also produce greater side effects and likely less brain barrier crossover per unit than lithium orotate, for the present invention about 0.05-5 mg and 400 micrograms per serving in a preferred embodiment per serving.

Preferred embodiments add additional antioxidants. Not wishing to be held to particular theory in free radical oxidation, a byproduct of oxygen species accumulation in tissue reacts negatively charged oxygen seeking to replace the electron it loses with surrounding tissue constituents, creating inflammation and damage as it extracts electrons from its surrounding environment. Amyloid formation in Alzheimer's disease for example is believed to be adversely affected by and in part may be induced by oxidative stress. The discoveries of the present invention may result because the brain is more sensitive to oxidative stress during periods of prolonged and or increased stimulation and metabolic activity, and the combination of low glycemic fuels and antioxidants reduces oxidative stress. A preferred embodiment of the present invention adds one or more of vitamin C, E, blueberry anthocyanins, cocoa flavanols, olive oil extract, or carotenoids such as algae astaxanthin to the chlorogenic acids, catechins and EGCG catechins of its blend of caffeine, coffee, tea and their derivatives.

A preferred embodiment of the present invention adds a select blend of non-psilocin or psilocybin mushroom extracts. While some studies have demonstrated a variety of neurological, and anti-inflammatory or toxicity reducing effects on metabolism and general inotropic effects in animal studies, as well as cognitive benefits in human trials whether these are translatable directly to the present invention is unknown.

Not wishing to be held to a particular theory mushrooms and or their extracts of the present invention, including particularly two or more of lions mane, reishi, maitake, enotake and cordyceps, are discovered to be a particularly effective blend for the present invention that may in part additively contribute to the enhanced anxiolysis, sleep induction and quality, accelerated recovery from sleep deprivation, and feelings of well-being seen with preferred embodiments. This more profound sense of well-being, present up to ten or more hours later, facilitates an easy onset and transition as desired to sleep reducing or preventing the usual difficulty and even insomnia typically prevalent with caffeine and particularly rekey i've and daily dose high caffeine or other brain stimulant use, and the eventual fatigue leading to sleep transition lacks the anxiety and feelings of “restlessness and difficulty falling asleep” and other deleterious side effects post caffeine use with prior art drinks, beverages or supplements. The present invention in fact is surprisingly discovered to further promote and extend the benefits of mental alertness with less side effects than previously possible, including the 1 correlated sense of well-being and enhanced awareness during periods of rest, helps to promote a relaxed transition to sleep and more fitful sense of rest and well-being on arising that are palpable and where one or more mushroom extracts may contribute to additional health additive benefits of the present invention particularly with regular use including cognitive benefits. In some embodiments may include anywhere from two to 18 mushroom extracts, and in a mist preferred embodiment 12, including lions' mane, reishi, maitake, enotake, chaga, and cordyceps, where a range from about 250 mg minimum to 2000 mg of total extract are believed key to fully enhancing brain health and systemic benefits of the present invention.

A profound sense of well-being, facilitated transition to a restful induced sleep, a more rapid recovery from a deeper more fitful sleep discovered, are then believed to be more profound when mushrooms or mushroom extract blends of the present invention are added as preferred embodiments. It is further discovered this greater sense of well-being and recovery to a restful state occurs requiring significantly fewer hours of sleep, often only 3-4 hours despite prolonged hours of wakefulness over a 24-hour period on occasion provided not attempted on a regular basis. This profound tranquility, relaxed state, and calmness both after use and on arising after sleep may include heightened awareness of one's surroundings and attentiveness and receptiveness to positively interact with others. This may manifest by including affect, mood, or other behavioral detail of others, as a generality enhanced often profound perceptive awareness allowing one to be undistracted in the moment particularly effectively. Preferred embodiments are virtually predicted to include additional benefit from inclusion of one or more micro doses of psilocin or psilocybin containing mushrooms or extracts at micro dose levels below the threshold of hallucinogenic induction when legally available as non-prescription food or nutritional supplements, and virtually suggested most effectively if followed by a positive interpersonal series of designed interactions or cognitive tests and exercises.

These and other benefits of the present invention are virtually discovered to remain without tachyphylaxis with regular use. They may include their delivery as a nutritional supplement or nootropic beverage. Delivery as a supplement may include replacement of the roasted coffee bean liquid coffee with caffeine powder in such supplements. Similarly, such replacement may be included non-coffee flavored preferences of the present invention as a nootropic beverage.

The present invention discovers toxicity to brain metabolism induced by caffeinated drinks, beverages, and supplements is related to need for optimized brain metabolic fuels; and toxicity is thereby with the addition of one or more of reduced and or eliminated; where these positive effects of the novel discovered energy require one or more of a low glycemic glucose stable carbohydrate; and or one or more of ketones and proteins, in a preferred embodiment hydrolyzed collagen, plant based, nut milk, pea, oat milk, whey protein or in a preferred embodiment a combination of at least two of the above protein sources. In addition, other aspects of preferred embodiments containing one or more of added non-coffee or tea antioxidants, micro dose lithium, and one or more of lion's mane, reishi, maitake, enotake, and cordyceps mushrooms or their fruit or mycelia extracts further contribute to its discovered novelty and effectiveness.

The present invention further discovers these benefits of optimized fuel or fuels and other additives of the present invention to caffeinated drinks, beverages, and or nootropic supplements are particularly enhanced by how caffeine is sourced; that caffeine derived from roasted coffee beans may be medium roast or deep roast but medium roast is preferred or caffeine powder, and preferably as a blend with one or more of green coffee bean extract, green tea, green matcha tea or green matcha tea powder more preferred; where cold brew of roasted and particularly medium roasted coffee beans is preferred as the roast coffee bean source, still more preferably cold brew of roasted beans, and most preferably cold brew of roasted beans by a drip method, all of which is preferred or caffeine powder alone as the sole caffeine source; that any source if caffeine has greater spread of absorption vs time with one or more of green coffee bean extract and green tea; and in a preferred embodiment the most well tolerated caffeine combination when both green coffee bean extract and matcha tea are added to a caffeine powder or coffee source adding their own caffeine as well as their own antioxidants; that D-ribose, honey (fructose, maltose, sucrose) provides added benefit as an immediate low glycemic fuel source for brain metabolism; that 5 g hydrolyzed collagen further enhances and prolongs alertness, that 2.5 g medium chain triglycerides dies so still further; that hydrolyzed collagen increased in addition to 15 g does so still further; and that adding 9 g of collagen peptides does so still further; adding one or more of micro dose lithium, additional antioxidants and mushroom extracts does so still further.

Not wishing to be held to particularly theory it is believed the present invention may induce its profound benefits to sustained mental focus and sense of well-being by optimizing brain metabolism through bus combinations and a varieties of combinations thereby improved, some of which may be unknown presently and remain to elucidated or more fully elucidated, and provides multiple benefits individually surprising and cumulatively never before possible with prior art; with multiple brain and systemic health benefits prophetically predicted and others unknown; where induced benefits include but are not limited to:

Stabilization of glucose levels, with addition of low glycemic fuels that eliminate need for high glycemic sugars and eliminate toxicity mid 0 fuel additive artificial sweeteners;

Reduction or elimination of toxic byproducts of metabolic fatigue during prolonged sustained brain metabolism of the present invention due to availability of glucose or alternative energy sources throughout most or all of the hours of caffeine brain stimulant delivery;

Further reduction or elimination of toxic byproducts of metabolic fatigue during prolonged sustained brain metabolism of the present invention due to availability of an antioxidant blend throughout most or all of the hours of caffeine brain stimulant delivery;

Further reduction or elimination of toxic byproducts of metabolic fatigue during prolonged sustained brain metabolism of the present invention due to availability of an antioxidant blend throughout most or all of the hours of caffeine brain stimulant delivery;

Further reduction or elimination of toxic byproducts of metabolic fatigue during prolonged sustained brain metabolism of the present invention due to availability of micro dose lithium reducing formation of inflammatory metabolites and or reducing baseline fatigue through beneficial improvement of mental affect;

Its natural low-calorie appetite suppression due to its nutrient density, supplying the brain with its enhanced nutritional requirements following brain caffeine stimulation adds potential further metabolic optimization. Ingestion of high glycemic sugars and or foods high in saturated fats (fast foods) occurring during caffeine brain stimulated periods could slow brain metabolism with reduction of alertness and increase metabolic toxicity. Appetite suppression reduces these uncontrolled variables.

That the sum total of the novel additives, blends, and benefits of the present invention not only enhance alertness but contributed a profound sense of well-being that can improve sleep, rest, overall energy;

That the sum total of the novel additives, blends, and benefits of the present invention not only enhance alertness but contributed a profound sense of well-being that can alleviate or reduce depression, mood instability, suicide ideation, and suicide incidence and prevalence for the duration of its daily use;

That the sum total of the novel additives, blends, and benefits of the present invention with regular use may not only enhance alertness but reduce cognitive decline for the duration of its daily use;

That the sum total of the novel additives, blends, and benefits of the present invention with regular use may not only enhance alertness but enhance cognition, including short term memory for the duration of its daily use;

That the sum total of the novel additives, blends, and benefits of the present invention may not only enhance alertness but improve cognition and reduce the mental deficits of dementias and or Alzheimer's for the duration of its daily use as well as reduce histopathologic changes associated with these conditions;

A new class of beverage is surprisingly discovered by the present invention that delivers caffeine as an extended release, not just quick onset, and provides the equivalent of extended release simultaneously delivered low glycemic brain preferred healthy sources of fuel for brain metabolism: which in preferred embodiments may include variable onset antioxidant delivery. and similarly, a blend of mushroom extracts that promote brain and systemic health. One or at most two servings a day provides nourishing, sustainable hours of alertness and refueling of the brain with essential nutrients not possible with prior art. The brain fuels provide the dual benefit of short term appetite suppression due to satiety from about 200 nutrition packed calories with 11 grams of short hydrolyzed collagen and long (nut milk, or milk protein) duration of absorption blend for both brain fuel and amino acid precursors for receptor neurotransmitters, 2.5 grams of medium chain triglyceride fatty acids as ketones for ideal long term brain energy loaded with antioxidants but only about 6 grams of low glycemic sugar.

Reduced meal ingestion, and particularly reduced sugar and or fatty meal cravings, are discovered to facilitate and prolong sustainable benefits of the discovered low glycemic blend brain fuel and antioxidants of the present invention otherwise reduced by nutritional excessive ingestion of either.

The present invention unexpectedly discovers compositions and means to enhance mental focus, works for prolonged periods of time enabling intense concentration, and is virtually anticipated can enhance cognitive function, including memory, reduced rate of cognitive decline with regular use, and reverse it in some cases; by a combination of:
1) compositions to spread out caffeine absorption more evenly over short, medium, and longer duration;
2) simultaneously optimizing fuel delivery with a short, medium and long duration fuel source with other beneficial effects on brain function such as glucose level stabilization;
3) similarly providing potential differential absorption of a blend of antioxidants from quick to delayed absorption;
4) a selective blend of mushroom extracts that add numerous potential brain and systemic complimentary health, sleep, and feelings of wellness; and
5) micro-dosed lithium versus high doses used to treat bipolar disorders, with regular use as in water supplies with micro doses higher levels of lithium, demonstrates reduced suicide rates, may reduce depression, and help facilitate improved brain test and physiology supportive of a baseline level of reduced “noise” or “mental thought” obsession, where any added tranquility via reduced background mental “thoughts” may facilitate improved alertness as well.

The present invention is the first to offer health generating improved mental focus, alertness, and sustainable concentration on a daily basis for the majority if not entirety of a workday or even the entire practical timeframe of wakefulness: 16 hours−minus about 2 hours for arising, going to sleep and 2 hours in between on two maximum servings per day delivering up to 12 hours of never before seen benefit.

Not wishing however to be held to particular theory, Breinfuel™ is designed to provide differential rates of absorption and blood brain barrier entry to cerebral circulation of caffeine sources blends and low glycemic glucose stable fuel or fuels to create a gradual, sustained, long duration synchronization of stimulant concentration and fuel versus time; low glycemic brain fuel concentration versus time; caffeine concentration versus time; antioxidant concentration versus time; along with additives including one or more of non-caffeine sources added antioxidants; added micro dose lithium, and or added mushroom, mushrooms, extract or extract blend, all designed amplify the positive alertness and potential cognitive benefits of each individually by increasing availability of inotropic chemicals that facilitate brain health and or reduce toxicity of its metabolism particularly with a caffeine sources brain stimulant. Coffees and teas, with their potential caffeine related health benefits become realized in ways not before possible.

The surprising results of use of embodiments of the present invention include onset of a phase of enhanced alertness over about thirty minutes that is almost imperceptibly smooth, without any evidence of systemic stimulation; mental effort quickly thereafter becomes effortless, and enhanced on an as needed basis such as to provide intense mental concentration, such as working on a presentation or other project, and becomes highly sustainable for many hours.

A tranquil feeling of calm that juxtaposes its use, contributed to ease of sleep onset, and follows sleep on arising are key additional benefits of the present invention versus prior art. if sleep use of the present supports the incredible alertness and focus; no brain stimulant, energy drink, nootropic drug, coffee, tea, or other brain supplement provides or could be predicted to provide the surprising combination of relaxation and tranquility induced by the present invention with the enhanced mental energy and focus sustained for many hours as well by the present invention.

Despite the above advantages use of preferred embodiments of the present beyond two servings a day with total caffeine levels of 100-400 multisource mg per day per serving; and 0-400 mg of ECGS per day per serving; where two servings allow benefits over much of a 16 hour non sleep cycle; would not be advised, could add potential toxicity. Use during pregnancy, breast feeding, in presence of thyroid disease, use of blood thinners, or for diabetics or individuals on other medications should first be approved by their physician.

It is a virtual discovery of the present invention that multiple adverse effects of toxins, pollutants, pesticides, heavy metals and other contaminants are in many cases reduced by regular use of the present invention.

It is a virtual discovery of the present invention that the immune system may be enhanced by inotropic immuno-modulators, that reduced immunity become improved, and that autoimmune diseases may be decreased incidence, where present in some case decreases in severity, and in other cases subjects afflicted may respond in improved ways to medications for their treatment.

It is a virtual discovery of the present invention that incidence of cancer may be decreased, life expectancy in presence of others increased, effectiveness of treatments of some cancers enhanced, and adverse effects of chemotherapy in some case reduced.

It is a commonly believed that cognitive decline is accelerated by environmental toxins, pollutants, pesticides, leeched plastics, nutritionally inadequate diets and excessive unhealthy components that have tainted or food supply and together contribute to accelerated cognitive decline with age particularly in individual cars of sensitivity to one or more of these toxins, and more particularly with jess robust, weak, or compromised immune systems, where the hallmark of cognitive decline most prevalent is Alzheimer's disease. Just as good nutrition fosters general overall good systemic health and is a multifactorial disease preventative, Breinfuel™ optimizes brain and systemic health in ways not previously possible and is virtually predicted to help prevent cognitive decline, to reduce the degree of cognitive loss, and repair the histopathologic changes if Alzheimer's and other dementias with regular use. While long term peer reviewed study to prove these advantages have a high cost beyond the scope of this invention, they are virtually anticipated. It is virtually predicted that like a nutritional food supplement Breinfuel™ will provide protection reducing the rates of cognitive decline and may help reverse that decline. But like a nutritional supplement and unlike a curative drug to optimally maintain these and other health benefits that may accrue will be most facilitated with regular use.

In one embodiment, the present invention is directed to a composition comprising a caffeinated blend of coffee(s), coffee powder and teas, low glycemic fueled blend of medium chain triglycerides optionally facilitated by D-ribose, and proteins, and in a preferred embodiment micro-dose lithium, added antioxidants, and a mushroom extract blend, facilitate never before experienced juxtaposed relaxed tranquil state of awareness with greater mental alertness, concentration and intense focus sustainable for prolonged periods, yet ease of transition to a state of recovery and ease of transition to a fitful deep and restful sleep and recovery over shorter time frames, with a feeling of profound well-being on arising.

A most preferred embodiment of the present invention is directed to a composition comprising per 8 ounce serving:

2.12 ounces cold brewed drip French roast (200 mg caffeine);
600 mg green coffee bean extract with 300 mg chlorogenic acid (20 mg caffeine);
4 g green matcha tea powder with catechins and ECG (65 mg caffeine);
5.5 g D ribose;
17 g honey (fructose, maltose, glucose, sucrose);
16 g hydrolyzed collagen;
9 g collagen peptides;
2.5 g medium chain triglycerides (C8 preferred and ircC8 and C10);
350 mcg lithium orotate;
1 g vitamin c;
5 mg astaxanthin;
125 mg resveratrol;
40 mg quercetin;
10 mg fisetin;
4 g blueberry powder;
540 mg reishi mushroom extract;
540 mg lion's mane mushroom extract;
540 mg cordyceps mushroom extract;
1 g 14 other extracts including preferred royal blazei, maitake, chaga, and enotake; and Sweeteners, stabilizers.

In other preferred embodiments the above is modified as follows:

cold brewed coffee is replaced with
Caffeine powder (200 mg)+
Flavored protein drink (5 g vegan)+
Hydrolyzed collagen reduced (15 to 11 g)

The Applicant has unexpectedly discovered the following:

1. It is a finding of the present invention that alertness is sustained for prolonged intervals, typically 6-9 hours, without the shakiness, jitters or other signs of systemic high caffeine dosing despite ingestion of 150-350 mg of caffeine in one serving;
2. It is a finding of the present invention that the sustained alertness that results allows driving at night particularly in poorly lit conditions for prolonged periods of several hours despite initial states of fatigue;
3. It is a finding of the present invention that cravings for high glycemic foods such as desserts and sweetened beverages harmful to alertness and optimal brain function are suppressed within 30-45 minutes following ingestion;
4. It is a further finding of the present invention that cravings for high levels of saturated fats such as fried foods harmful to alertness and optimal brain function are suppressed within 30-45 minutes following ingestion;
5. It is a further finding of the present invention that with regular daily use of a preferred embodiment cholesterol levels significantly decrease;
6. It is a further finding of the present invention that with regular daily use of a preferred embodiment low density lipoprotein levels significantly decrease;
7. It is a further finding of the present invention that with regular daily use of a preferred embodiment high density lipoprotein levels significantly increase;
8. It is a further virtual finding of the present invention that with regular daily use of a preferred embodiment decreases and effects of ischemic cerebral stroke are reduced;
9. It is a further virtual finding of the present invention that with regular daily use of a preferred embodiment risk of a cardiac event and or specifically myocardial infarction statistically significantly is decreased;
10. It is a still further virtual finding of the present invention that with regular daily use of a preferred embodiment inflammation is decreased and that serum measures of erythrocyte sedimentation rate (ESR), C-reactive protein (CRP) and plasma viscosity (PV) are statistically significantly decreased;
11. It is a still further virtual finding of the present invention that with regular daily use of a preferred embodiment the Incidence of cancer assessed over a large population is statistically significantly decreased;
12. It is a still further virtual finding of the present invention that with regular daily use of a preferred embodiment one or more cancers has statistically significantly improved 5-year survival results on chemotherapy;
13. It is a still further virtual finding of the present invention that with regular daily use of a preferred embodiment treatment of one or more cancers has statistically significantly reduced side effects on chemotherapy;
14. It is a still further virtual finding of the present invention that with regular daily use of a preferred embodiment diabetic glucose control improves;
15. It is a still further virtual finding of the present invention that with regular daily use of a preferred embodiment the incidence of adult onset type II diabetes is statistically significantly decreased:
16. It is a still further virtual finding of the present invention that with regular daily use of a preferred embodiment the incidence of post-traumatic stress syndrome, or panic attacks is statistically significantly decreased;
17. It is a still further virtual finding of the present invention that with regular daily use of a preferred embodiment the incidence of diagnosis and or manifestations of bipolar disorder and or degree of drug therapy needed is statistically significantly decreased;
18. It is a still further virtual finding of the present invention that with regular daily use of a preferred embodiment the incidence and or degree of depression is statistically significantly reduced;
19. It is a still further virtual finding of the present invention that with regular daily use of a preferred embodiment the incidence of suicide assessed in large populations is statistically significantly reduced;
20. It is a still further virtual finding of the present invention that with regular daily use of a preferred embodiment the incidence of excessive Intestinal mucosal post capillary venue vasodilation becomes significantly reduced;
21. It is a still further virtual finding of the present invention that with regular daily use of a preferred embodiment the flora of the biome in the intestinal tract becomes improved;
22. It is a still further virtual finding of the present invention that with regular daily use of a preferred embodiment the incidence of hepatic toxicity and or its degree is statistically significantly improved;
23. It is a finding of the present invention that the relaxed state of alertness is effortless, occurring without intention, or effort to concentrate harder, based only on whatever is required reflexively in the moment or for the task at hand;
24. It is a further finding of the present invention that the rested, relaxed state of well-being induced during sleep is maintained for up to 36 hours from time of ingestion;
25. It is a still further finding of the present invention that the rested, relaxed state of well-being of the present invention is greater in embodiments that include mushroom extracts;
26. It is a still further finding of the present invention that these extracts include lion's mane;
27. It is a still further finding of the present invention that these extracts include lion's mane, reishi, chaga, maitake, enoki, and cordyceps;
28. It is a still further finding these mushroom extracts may include one or more of royal blazei, Mesima, Turkey tail, Oyster Mushroom, Artists conk. Agarikon, Amidou, and or Shiitake, for optimal ease of falling asleep four or more hours after ingestion when desired and arising with a restful, relaxed feeling of well-being;
29. It is a further finding of the present invention that the onset of alertness effect is typically 45-60 minutes; duration typically 5-9 hours; and feeling of well-being onset of 2-4 hours and sustained for at least 12 hours;
30. It is a virtual finding of the present invention that in a population of early and or moderately cognitively impaired subjects in a preferred embodiment the rate of cognitive decline decreases versus controls with statistical p<0.1 significance and in some studies p<0.05; 31. It is a virtual finding of the present invention that in a population of cognitive impaired subjects diagnosed with early and or moderate Alzheimer's in a preferred embodiment the rate of cognitive decline decreases versus controls with statistical p<0.1 or p<0.05 significance;
32. It is a further virtual finding of the present invention that in a population of cognitive impaired subjects diagnosed with early and or moderate level Alzheimer's, in a preferred embodiment the degree of cognitive impairment decreases versus controls with statistical p<0.1 and or p<0.05 significance;
33. It is a further virtual finding of the present invention that in a population of cognitive impaired subjects diagnosed with some forms of severe dementia and or severe Alzheimer's in a preferred embodiment the rate of cognitive decline decreases and or of cognitive impairment decrease versus controls with statistical p<0.1 and or p<0.05 significance with periods of use of four months or greater.
34. It is a further virtual finding of the present invention that it induces increasing mental focus, clarity of thought, anxiolysis with profound reduction of stress and anxiety, relaxation, improved onset and quality of sleep, and an overall greater feeling if wellbeing.

Compositions of the present invention including Breinfuel™ utilize several co-amplifying discoveries to create its unique combination of mental focus, sustained concentration, appetite and high glycemic source suppression, fitful restful sleep, and feelings of well-being, in a preferred embodiment:

I a Combination of Caffeine Sources and their Derivatives—Two or More of:
Caffeine powder,
Roasted coffee bean coffee,
Green coffee bean extract, and or
Green tea/matcha tea; and

### II Two or More of Low Glycemic Fuels:

D ribose;
MCT oil ketones, and or omega 3 fatty acids; and
Protein—where in a preferred embodiment protein includes
collagen and more preferably hydrolyzed collagen; and or in addition one or more of
nut milk extract, pea milk, or oat milk;
milk whey protein or vegetable protein;

Preferred Embodiments of the present invention further comprise:

### III Lithium

Lithium orotate, 25-90 mcg

### III Additional Antioxidant Blend

One or more of:
Fruit-based extracts such as tart cherry juice, blueberry powder anthocyanins,
Cocoa flavanols,

### Resveratrol,

### Vitamin C,

### Vitamin E, and or

Olive oil or extract.

### IV Mushroom Extract Blend

mushroom blend extract—
one or more of lion's mane, reishi, enoki, and cordyceps,
optionally maitake, chaga, royal blazei
optionally others

### V Probiotic

### 10,000-50,000 CFU

5-50 colonies

The Applicant has unexpectedly discovered that consumption of compositions of the present invention result in:
ease of sleep induction;
feeling of relaxed, well-being;
effortless sustained concentration;
no jitters or shakiness;
no crash or brain fog;
appetite suppression general;
reduced cravings for high glycemic sugars/carbohydrates;
enhanced short term memory
improved ability to perform tasks requiring intense mental concentration
improved ability to retain information via short- and long-term memory

The pairing of caffeine & derivatives and brain fuel levels it delivers sustained release for hours of both is unique, surprising, and the effects are industry disruptive:

Compositions providing release of caffeine and sources of brain fuel being further enhanced by a preferred embodiment's additional flavanols, anthocyanins and related antioxidants; mushroom extract blend consisting of a spectrum of:

### Improving Cognitive Impairment

General dementia and the specific clinical and histologic findings of Alzheimer's disease have become increasingly prevalent over the last 100 years. Multiple hypotheses for Alzheimer's disease causation alone include:

Amyloid formation (amyloid beta peptide (Aβ) in extracellular deposits termed senile plaques);
Tau (protein leading to microtubule formation;
Choline (reduction of acetylcholine);
Oxidative stress (may be causative of reactions producing amyloid and or tau)
Breinfuel™ combines caffeinated derivatives known to facilitate brain function and used as a precursor for cognitive drugs, and preferred low glycemic brain fuels, positively impacts brain physiology and overall health, surprisingly and unexpectedly when all are ingested simultaneously can be optimized to provide quick onset, enhanced peak effectiveness, and profound longer durations of sustained mental focus and intense concentration.

This surprising discovery revolutionizes the way we can use and synchronize caffeinated coffee and tea derivatives, in a preferred embodiment “paired” via a “blend” of each—in one preferred embodiment three sources of caffeine and derivatives with three sources of brain fuel. This profoundly enhanced ability to virtually effortlessly sustain prolonged intense periods of focus and concentration revolutionize and improve or define a new category of “energy drinks” via powerful cognitive enhancement and sustained mental focus alertness.

A preferred embodiment that includes caffeinated derivatives of powerful antioxidants as well as other selected antioxidants, mushroom extracts, and low glycemic optimized “brain fuels” offer potential for therapeutic value with daily use that not only add cognitive value, but promote multi-system health benefits that may include one or more of oncologic, immune-modulating, cardiovascular, endocrine, hepatocellular, and digestive health benefits. It may, in conjunction with oncologic drugs reduce their side effects or further increase their efficacy. Not wishing to be held to any particular theory it may delay, and or at least partially or more completely reverse the progressive cognitive decline of early or more advanced general dementia or Alzheimer's disease. As with most nutritionally derived benefits these advantages may be most completely realized with regular use and fall-off or be reversed with their prolonged absence.

Breinfuel™ in a preferred embodiment provides a nutrient dense power packed 190-250 calories, a blend of coffee and tea caffeinated derivatives, MCT oil ketones, collagen, almond extract proteins and minerals, mushroom extract with one or more of polysaccharides, polyphenols, proteases, terpenoids, lectins, illudins, ribosome inactivating proteins, vitamins and minerals a host of minerals, D-ribose, a potent blend of caffeinated derivatives and other antioxidants with modest amounts of honey and erythritol for sweetness.

It is another surprising discovery of the present invention that combining roasted coffee bean coffee (cold brew or hot) with a vegan milk source (almond, cashew, oat, hemp, or pea as examples) provides a liquid formulation within which this complex of ingredients remains stable with an acceptable commercial shelf life either at 5 C or 22 C.

The following Examples are provided solely for illustrative purposes and are not meant to limit the invention in any way.

### Examples: Include Absorptive Order Options

As a hypothetical virtual illustration of pairing possible with three variable onset caffeine and derivative coffees and teas, with three variable onset low glycemic fuel sources from as-ribose to ketones to two forms of protein one of many combination pairings by which the synchronization of potent caffeinated derivatives and their antioxidants with low glycemic brain fuels is illustrated:
1. the initial fuel phase, where medium roast (cold brew drip) caffeinated coffee derivatives reach the brain along with quickly absorbed D-ribose sugar provide an onset with pairing of a caffeine and fuel source to the brain;
2. the intermediate time phase pairs green coffee bean extract caffeine and its slower release triggered via its CGA antioxidants along with MCT fatty acid ketones absorbed and crowding the blood brain barrier at about a similar frame for brain energy delivered fuel; and
3. the late onset prolonged duration phase that pairs for example matcha tea extract caffeine and it's release via its catechin and EGCG antioxidants along with more slowly broken down and absorbed collagen and almond nut extract protein (to concentrated glycine and other amino acids enhancing brain metabolism) as fuel reaching the brain as the third timed phase of caffeine-fuel source pairing.

Other advantages result from these pairings as well:

4. The low glycemic index high protein drink that results avoids glucose spikes, helps to stabilize glucose levels to which brain function is very sensitive, and adds powerful antioxidants and a blend of mushroom extracts where multiple compounds with brain and other health benefits have been isolated including polyphenols, polysaccharides, proteases, terpenoids, lectins, illudins, ribosome inactivating proteins, as well as multiple vitamins and minerals provides potential for additional brain and systemic health benefits; and
5. Having three caffeinated derivatives with their own individual absorption rates and three fuel sources with equally individualized absorption rates promotes improved pairings whatever their individual order as exemplified above, a key to smooth onset and effective induction as well as prolonged duration of sustained mental alertness and focus. Trying to achieve the same with quick onset roasted coffee/caffeine levels not coordinated with supportive optimal fuel or even lacking any low glycemic fuel source at all creates a short duration jittery level of alertness with poor recovery. As a result, Breinfuel™ is a uniquely optimized nootropic brain and health drink providing a concentrated nutrient dense drink that delivers varied onset caffeinated derivatives and low glycemic brain metabolism fuels for optimal pairing versus time range for absorption among other brain and health benefits.

Still other benefits to brain and multiple system health may accrue from a preferred Breinfuel™ formulation:

6. Breinfuel™ may be effective in reducing inflammation via its antioxidants and immune-modulating compounds part of its mushroom extract blend and antioxidants that can prolong more intense brain function;
7. It's nutrient dense mineral, and vitamins as well as MCT ketones and protein, may support better nutrition and portion allocation. For example, it is a natural appetite suppressant due to its nutrient density and ketone and protein fuel sources, that could help alleviate sugar cravings and lack of portion control that also adversely affect brain health and mental activity;
8. Heavy metals, fried foods, pesticides, pollutants and other toxins we absorb trigger oxidation as well, where the single electron oxygen radical of the oxygen species seeks out a second surrounding electron causing considerable chemical damage, inflammation and compromised function in the process. Amyloid beta peptide formation in Alzheimer's for example appears to be associated with inducing considerable oxidative stress and neuronal destruction. Rainwater's proprietary blend of antioxidants—caffeic and ferulic acids from roasted coffee beans; CGA's from unroasted coffee beans; catechins from matcha tea, the super antioxidant astaxanthin from red algae caretenoids pigment, anthocyanins from blueberry powder, multiple mushroom extract polysaccharides, and vitamin C combine with substantial quantities in every serving to combat and alleviate oxidative stress;
9. The goal is a gradual onset over about 60 minutes, with ability to naturally achieve effortless or intentionally targeted undistracted focus of even high intensity for the next several hours, with a palpable sense of calmness. Breinfuel™ provides a prolonged steady state and more comprehensive delivery of caffeinated coffee and tea derivatives than any cup of coffee with equally prolonged low glycemic b-fuels. After a single serving of 6 oz of Breinfuel™, as well as with regular or daily use, the brain appears becomes physiologically optimized based on performance, as a user become seamlessly mentally energized in profound ways, experiencing profound benefits from heightened alertness; and
10. No significant increase in blood pressure, heart rate, or blood glucose levels over duration of use.

### PREFERRED EMBODIMENTS

BW.pe 2.0: 180 mg caffeine, 10 g collagen and almond protein, 2.5 g MCT's

1. lithium (as orotate), 0.6 mg
2. cold brew coffee concentrate French roast, 1.06 oz 100 mg caffeine
3. green coffee bean extract, 800 mg,*15 mg caffeine
4. organic matcha tea, 2-4 g, total egcg content is 200-350 mg, 65 mg caffeine
5. mct ketones 2.5 g,
6. hydrolyzed collagen 5 g,
7. d-ribose, 5.5 g,
8. resveratrol, 500 mg
9. astaxanthin, 10 mg,
10. nature's way vitamin c, 1000 mg,
11. blueberry powder, 7.5 g,
12. almond nut conc, 22.5 g,
13. mushroom ex: (non-psilocybin or psilocyn)

a. lion's mane, 500-600 mg

b. reishi, 500 mg

c. cordyceps, 500 mg,

14. split gill polypore, 6 mg
15. probiotic 25 cfu (10-50) billion; 10 strains (5-20)
16. tart cherry juice conc 1-2 ml
17. honey 7.5-10 g,
18. erythritol 3-6 g
19. water distilled or sourced 6 (4-12) ounces nets 8 ounces

BW.pe 2.1: 285 mg caffeine 16 g collagen protein; 9 grams peptides; 2.5 g MCT's

1. lithium (as orotate), 0.35 mg
2. cold brew coffee concentrate French roast, 2.12 oz, 200 mg caffeine
3. green coffee bean extract, 800 mg, (50% chlorogenic acid), 20 mg caffeine
4. organic matcha tea, 4 g, total egcg content is 200-350 mg, 65 mg caffeine
5. mct ketones 2.5 g,
6. hydrolyzed collagen 16 g,
7. d-ribose, 5.5 g,
8. resveratrol, 250 mg
9. astaxanthin, 5 mg,
10. nature's way vitamin c, 1000 mg,
11. blueberry powder, 4 g,
12. mushroom ex: (non-psilocybin or psilocyn)

a. lion's mane, 500-600 mg

b. reishi, 500 mg

c. cordyceps, 500 mg,

d. enokitake, 500-600 mg,

e. chaga, 74 mg

f. maitake, 71 mg

g. Oregon reishi, 55 mg

h. royal sun blazei, 40 mg

i. mesima, 38 mg

j. turkey tail, 24 mg

k. oyster mushroom, 24 mg

l. artists conk, 15 mg

m. Oregon reishi, 15 mg

n. agarikon, 15 mg

o. amidou, 10 mg

p. shiitake, 10 mg

q. maitake ii, 8 mg

r. birch pore, 6 mg

s. split gill polypore, 6 mg

13. probiotic 25 cfu (10-50) billion; 10 strains (5-20)
14. honey 2 tbsp,
15. erythritol 3-6 g
16. water distilled or sourced 8 ounces

Extract, blend, preferably served chilled. Makes a 10 oz serving

BW.pe 2.2: 285 mg caffeine; 15 g collagen protein; 9 g peptides; 2.0 g MCT's

1. lithium (as orotate), 0.35 mg
2. cold brew coffee concentrate French roast, 2.12 oz, 200 mg caffeine
3. green coffee bean extract, 600 mg, (50% chlorogenic acid), 15 mg caffeine
4. organic matcha tea, 4 g, total egcg content is 200-350 mg, 65 mg caffeine
5. mct ketones 2.0 g,
6. hydrolyzed collagen 12.8 g,
7. collagen peptides 7.2 g,
8. d-ribose, 5.75 g,
9. resveratrol, 250 mg
10. astaxanthin, 5 mg,
11. nature's way vitamin c, 1000 mg,
12. blueberry powder, 4 g,
13. mushroom ex: (non-psilocybin or psilocyn)

a. lion's mane, 500-600 mg

b. reishi, 500 mg

c. cordyceps, 500 mg,

d. enokitake, 500-600 mg,

e. chaga, 74 mg

f. maitake, 71 mg

g. Oregon reishi, 55 mg

h. royal sun blazei, 40 mg

i. mesima, 38 mg

j. turkey tail, 24 mg

k. oyster mushroom, 24 mg

l. artists conk, 15 mg

m. Oregon reishi, 15 mg

n. agarikon, 15 mg

o. amidou, 10 mg

p. shiitake, 10 mg

q. maitake ii, 8 mg

r. birch pore, 6 mg

s. split gill polypore, 6 mg

14. probiotic 25 cfu (10-50) billion; 10 strains (5-20)
15. honey 2 tbsp,
16. erythritol 3-6 g

water distilled or sourced 8 ounces, makes 10 ounces

BW.pe 2.3: 385 mg caffeine; 9 g collagen protein; 8.8 g peptides; 2.0 g MCT's antiox med

1. lithium (as orotate), 0.35 mg
2. cold brew coffee concentrate French roast, 2.12 oz, 200 mg caffeine
3. caffeine powder, 100 mg
4. green coffee bean extract, 600 mg, (50% chlorogenic acid), 15 mg caffeine
5. organic matcha tea, 4 g, total egcg content is 200-350 mg, 65 mg caffeine
6. mct ketones 2.0 g,
7. hydrolyzed collagen 12.8 g,
8. collagen peptides 7.2 g,
9. d-ribose, 5.75 g,
10. resveratrol, 250 mg
11. astaxanthin, 5 mg,
12. nature's way vitamin c, 1000 mg,
13. blueberry powder, 4 g,
14. mushroom ex: (non-psilocybin or psilocyn)

a. lion's mane, 500-600 mg

b. reishi, 500 mg

c. cordyceps, 500 mg,

d. enokitake, 500-600 mg,

e. chaga, 74 mg

f. maitake, 71 mg

g. Oregon reishi, 55 mg

h. royal sun blazei, 40 mg

i. mesima, 38 mg

j. turkey tail, 24 mg

k. oyster mushroom, 24 mg

l. artists conk, 15 mg

m. Oregon reishi, 15 mg

n. agarikon, 15 mg

o. amidou, 10 mg

p. shiitake, 10 mg

q. maitake ii, 8 mg

r. birch pore, 6 mg

s. split gill polypore, 6 mg

15. probiotic 25 cfu (10-50) billion; 10 strains (5-20)
16. honey 2 tbsp,
17. erythritol 3-6 g

water distilled or sourced 8 ounces, makes 10 ounces

BW.pe 2.4: 340 mg caffeine; 12.8 g collagen protein; 7.2 g peptides; 2.0 g MCT's antiox med

1. lithium (as orotate), 0.35 mg
2. cold brew coffee concentrate French roast, 2.12 oz, 180 mg caffeine
3. caffeine powder 75 mg
4. green coffee bean extract, 600 mg, (50% chlorogenic acid), 15 mg caffeine
5. organic matcha tea, 4 g, total egcg content is 200-350 mg, 65 mg caffeine
6. mct ketones 2.0 g,
7. hydrolyzed collagen 12.8 g,
8. collagen peptides 7.2 g,
9. d-ribose, 5.75 g,
10. resveratrol, 250 mg
11. astaxanthin, 5 mg,
12. nature's way vitamin c, 1000 mg,
13. blueberry powder, 4 g,
14. mushroom ex: (non-psilocybin or psilocyn)

a. lion's mane, 500-600 mg

b. reishi, 500 mg

c. cordyceps, 500 mg,

d. enokitake, 500-600 mg,

e. chaga, 74 mg

f. maitake, 71 mg

g. Oregon reishi, 55 mg

h. royal sun blazei, 40 mg

i. mesima, 38 mg

j. turkey tail, 24 mg

k. oyster mushroom, 24 mg

l. artists conk, 15 mg

m. Oregon reishi, 15 mg

n. agarikon, 15 mg

o. amidou, 10 mg

p. shiitake, 10 mg

q. maitake ii, 8 mg

r. birch pore, 6 mg

s. split gill polypore, 6 mg

15. probiotic 25 cfu (10-50) billion; 10 strains (5-20)
16. honey 2 tbsp,
17. erythritol 3-6 g

water distilled or sourced 8 ounces, makes 10 ounces

BW.pe 2.5: 315 mg caffeine; 12.8 g collagen protein; 7.2 g peptides; 2.0 g MCT's, antiox hi

1. lithium (as orotate), 0.35 mg
2. cold brew coffee concentrate French roast, 2.12 oz, 180 mg caffeine
3. caffeine powder, 55 mg caffeine
4. green coffee bean extract, 600 mg, (50% chlorogenic acid), 15 mg caffeine
5. organic matcha tea, 4 g, total egcg content is 200-350 mg, 65 mg caffeine
6. mct ketones 2.0 g,
7. hydrolyzed collagen 12.8 g,
8. collagen peptides 7.2 g,
9. d-ribose, 5.75 g,
10. resveratrol, 250 mg
11. astaxanthin, 7.5 mg,
12. nature's way vitamin c, 1000 mg,
13. blueberry powder, 7.5 g,
14. mushroom ex: (non-psilocybin or psilocyn)

a. lion's mane, 500-600 mg

b. reishi, 500 mg

c. cordyceps, 500 mg,

d. enokitake, 500-600 mg,

e. chaga, 74 mg

f. maitake, 71 mg

g. Oregon reishi, 55 mg

h. royal sun blazei, 40 mg

i. mesima, 38 mg

j. turkey tail, 24 mg

k. oyster mushroom, 24 mg

l. artists conk, 15 mg

m. Oregon reishi, 15 mg

n. agarikon, 15 mg

o. amidou, 10 mg

p. shiitake, 10 mg

q. maitake ii, 8 mg

r. birch pore, 6 mg

s. split gill polypore, 6 mg

15. probiotic 25 cfu (10-50) billion; 10 strains (5-20)
16. honey 2 tbsp,
17. erythritol 3-6 g

water distilled or sourced 8 ounces, makes 10 ounces

BW.pe 2.6: 315 mg caffeine; 12.8 g collagen protein; 7.2 g peptides; 2.0 g MCT's, antiox hi

1. lithium (as orotate), 0.35 mg
2. cold brew coffee concentrate French roast, 2.12 oz, 180 mg caffeine
3. cold brew coffee concentrate mocha, 1.06 oz, 55 mg caffeine
4. green coffee bean extract, 600 mg, (50% chlorogenic acid), 15 mg caffeine
5. organic matcha tea, 4 g, total egcg content is 200-350 mg, 65 mg caffeine
6. mct ketones 2.0 g,
7. hydrolyzed collagen 12.8 g,
8. collagen peptides 7.2 g,
9. d-ribose, 5.75 g,
10. resveratrol, 250 mg
11. astaxanthin, 10 mg,
12. nature's way vitamin c, 1000 mg,
13. blueberry powder, 7.5 g,
14. mushroom ex: (non-psilocybin or psilocyn)

a. lion's mane, 500-600 mg

b. reishi, 500 mg

c. cordyceps, 500 mg,

d. enokitake, 500-600 mg,

e. chaga, 74 mg

f. maitake, 71 mg

g. Oregon reishi, 55 mg

h. royal sun blazei, 40 mg

i. mesima, 38 mg

j. turkey tail, 24 mg

k. oyster mushroom, 24 mg

l. artists conk, 15 mg

m. Oregon reishi, 15 mg

n. agarikon, 15 mg

o. amidou, 10 mg

p. shiitake, 10 mg

q. maitake ii, 8 mg

r. birch pore, 6 mg

s. split gill polypore, 6 mg

15. probiotic 25 cfu (10-50) billion; 10 strains (5-20)
16. honey 2 tbsp,
17. erythritol 3-6 g

water distilled or sourced 8 ounces, makes 10 ounces

BW.pe 2.7a (REG CAFF): 265 mg caffeine; 12.8 g collagen protein; 7.2 g peptides; 2.0 g MCT's, antiox hi

1. lithium (as orotate), 0.35 mg
2. cold brew coffee concentrate French roast, 1.57 oz, 135 mg caffeine
3. cold brew coffee concentrate mocha or another flavor, 1.06 oz, 55 mg caffeine
4. green coffee bean extract, 600 mg, (50% chlorogenic acid), 15 mg caffeine
5. organic matcha tea, 4 g, total egcg content is 200-350 mg, 65 mg caffeine
6. mct ketones 2.0 g,
7. hydrolyzed collagen 12.8 g,
8. collagen peptides 7.2 g,
9. d-ribose, 5.75 g,
10. resveratrol, 250 mg
11. astaxanthin, 10 mg,
12. nature's way vitamin c, 1000 mg,
13. blueberry powder, 7.5 g,
14. mushroom ex: (non-psilocybin or psilocyn)

a. lion's mane, 500-600 mg

b. reishi, 500 mg

c. cordyceps, 500 mg,

d. enokitake, 500-600 mg,

e. chaga, 74 mg

f. maitake, 71 mg

g. Oregon reishi, 55 mg

h. royal sun blazei, 40 mg

i. mesima, 38 mg

j. turkey tail, 24 mg

k. oyster mushroom, 24 mg

l. artists conk, 15 mg

m. Oregon reishi, 15 mg

n. agarikon, 15 mg

o. amidou, 10 mg

p. shiitake, 10 mg

q. maitake ii, 8 mg

r. birch pore, 6 mg

s. split gill polypore, 6 mg

15. probiotic 25 cfu (10-50) billion; 10 strains (5-20)
16. honey 2 tbsp,
17. erythritol 3-6 g

water distilled or sourced 8 ounces, makes 10 ounces

BW.pe 2.7b (HI CAFF): 315 mg caffeine; 12.8 g collagen protein; 7.2 g peptides; 2.0 g MCT's, antiox hi

1. lithium (as orotate), 0.35 mg
2. cold brew coffee concentrate French roast, 1.57 oz, 135 mg caffeine
3. cold brew coffee concentrate mocha or another flavor, 1.06 oz, 55 mg caffeine
4. caffeine powder, 50 mg
5. green coffee bean extract, 600 mg, (50% chlorogenic acid), 15 mg caffeine
6. organic matcha tea, 4 g, total egcg content is 200-350 mg, 65 mg caffeine
7. mct ketones 2.0 g,
8. hydrolyzed collagen 12.8 g,
9. collagen peptides 7.2 g,
10. d-ribose, 5.75 g,
11. resveratrol, 250 mg
12. astaxanthin, 10 mg,
13. nature's way vitamin c, 1000 mg,
14. blueberry powder, 7.5 g,
15. mushroom ex: (non-psilocybin or psilocyn)

a. lion's mane, 500-600 mg

b. reishi, 500 mg

c. cordyceps, 500 mg,

d. enokitake, 500-600 mg,

e. chaga, 74 mg

f. maitake, 71 mg

g. Oregon reishi, 55 mg

h. royal sun blazei, 40 mg

i. mesima, 38 mg

j. turkey tail, 24 mg

k. oyster mushroom, 24 mg

l. artists conk, 15 mg

m. Oregon reishi, 15 mg

n. agarikon, 15 mg

o. amidou, 10 mg

p. shiitake, 10 mg

q. maitake ii, 8 mg

r. birch pore, 6 mg

s. split gill polypore, 6 mg

16. probiotic 25 cfu (10-50) billion; 10 strains (5-20)
17. honey 2 tbsp,
18. erythritol 3-6 g

water distilled or sourced 8 ounces, makes 10 ounces

BW.pe 2.8 (HI CAFF): 385 mg caffeine; 4 g collagen protein, 5 g collagen peptides; 10.0 g MCT's, antiox hi

1. lithium (as orotate), 0.35 mg
2. cold brew coffee concentrate French roast, 1.57 oz, 135 mg caffeine
3. arabica & robusta bean roast coffee 300 mg caffeine
4. green coffee bean extract, 600 mg, (50% chlorogenic acid), 20 mg caffeine
5. organic matcha tea, 4 g, total egcg content 200-350 mg, 65 mg caffeine
6. mct ketones 9.0 g,
7. hydrolyzed collagen 4 g,
8. collagen peptides 5 g,
9. d-ribose, 5.75 g,
10. resveratrol, 250 mg
11. astaxanthin, 5 mg,
12. nature's way vitamin c, 500 mg,
13. blueberry powder, 7.5 g,
14. mushroom ex: (non-psilocybin or psilocyn)

a. lion's mane, 290 mg

b. reishi, 290 mg

c. cordyceps, 290 mg,

d. enokitake, 35 mg,

e. chaga, 74 mg

f. maitake, 71 mg

g. Oregon reishi, 55 mg

h. royal sun blazei, 40 mg

i. mesima, 38 mg

j. turkey tail, 24 mg

k. oyster mushroom, 24 mg

l. artists conk, 15 mg

m. Oregon reishi, 15 mg

n. agarikon, 15 mg

o. amidou, 10 mg

p. shiitake, 10 mg

q. maitake ii, 8 mg

r. birch pore, 6 mg

s. split gill polypore, 6 mg

15. probiotic 25 cfu (10-50) billion; 10 strains (5-20)
16. flavored syrups
17. cane sugar
18. erythritol 3-6 g
19. water distilled or sourced 8 ounces

makes 10-12 ounces

385 mg caffeine Brain Brew
Death Wish 300 mg caff: 8 oz from can
Leftcoast MCT grass coil acacia: 1 scoop
Superfoods matcha 65 mg caff: 2 level tsp
Vitacost GBCE 20 mg caff: 1.25 capsules
KAL Lithium Orotate: 0.1 capsules
Bulksupplement D-ribose: 1 level tsp
Life Extension resveratrol: 0.5 cap
Welikevitamins astaxanthin: 0.5 cap
Genius Mushroom: 1.5 caps
MyCommunity Host Defense: 1.5 caps
Nature'sWay Vit C 1 g with rose hips: 1 cap
Loov blueberry powder: 0.5 level tbsp

### Flavoring 5 FLAVORS

1 JordansSFreeDarkSltdEsp 1.5-3.5 tbsp

### +AND OR

### 2 Jordan Skinny Vanilla Carmel

### 3 Jordan Skinny Salted Carmel

### 3 Torani SF Salted Caramel

4 ToraniSyrupPeach90cal/sv (1-2 tbsp)

### 5 Torani SF S'Mores

Extract 1 min Blend 1 min Ninja IQ blender

### Chill

290 mg caffeine Brain Brew
Death Wish 300 mg caff: 3 oz from can
WanderingBear Cold brew: 5 oz*
Leftcoast MCT grass coil acacia: 1 scoop
Superfoods matcha 65 mg caff: 2 level tsp
Vitacost GBCE 20 mg caff: 1.25 capsules
KAL Lithium Orotate: 0.1 capsules
Bulksupplement D-ribose: 1 level tsp
Life Extension resveratrol: 0.5 cap
Welikevitamins astaxanthin: 0.5 cap
Genius Mushroom: 1.5 caps
MyCommunity Host Defense: 1.5 caps
Nature'sWay Vit C 1 g with rose hips: 1 cap
Loov blueberry powder: 0.5 level tbsp
Monk fruit: sweeten to taste (no corn syrup)
Honey: ¼-½ tbsp if needed
Extract 1 min Blend 1 min Ninja IQ blender

### Chill

*any 8 oz 150 mg caffeine coffee (mocha brew n bottle strother simpson)
Death Wish 300 mg caff: 8 oz from can
Leftcoast MCT grass coll acacia: 1 scoop Superfoods matcha 65 mg caff: 2 level tsp
Vitacost GBCE 20 mg caff: 1.25 capsules
KAL Lithium Orotate: 0.1 capsules
Bulksupplement D-ribose: 1 level tsp
Life Extension resveratrol: 0.5 cap
Welikevitamins astaxanthin: 0.5 cap
Genius Mushroom: 1.5 caps
MyCommunity Host Defense: 1.5 caps
Nature'sWay Vit C 1 g with rose hips: 1 cap
Loov blueberry powder: 0.5 level tbsp

### Flavoring 5 FLAVORS

1 JordansSFreeDarkSltdEsp 1.5-3.5 tbsp

### +AND OR

### 2 Jordan Skinny Vanilla Carmel

### 3 Jordan Skinny Salted Carmel

### 3 Torani SF Salted Caramel

4 ToraniSyrupPeach90cal/sv (1-2 tbsp)

### 5 Torani SF S'Mores

Extract 1 min Blend 1 min Ninja IQ blender

### Chill

Previous versions have somewhat comprised taste, agglutinatiob with precipitate on side walls of beverage contained within hours of being poured, and variable increased incidence of loose bowels, diarrhea, or stomach upset. Preferred embodiments 2.9 has eliminated these undesirable affects:

BW.pe 2.9 (HI CAFF): about 200 calories; 290-390 mg caffeine; 10 g protein, 11 g peptides; 7 g MCT's 5 g sugar carbohydrates

1. lithium (as orotate), 0.35 mg
2. cold brew coffee, 135 mg caffeine
3. espresso coffee shots, 120-220 mg caffeine
4. green coffee bean liquid extract, 75 mg, (50% chlorogenic acid), 5 mg caffeine
5. organic matcha tea liquid extract, 2 g, total egcg content 100-200 mg, 30 mg caffeine
6. mct sat fatty acids as liquid 7 g,
7. hydrolyzed collagen 10 g,
8. amino acids 11 g,
9. d-ribose, 5 g,
10. vitamin c, 1000 mg,
11. mushroom ex: (dried weight estimate)

a. lion's mane, 1.1 g

b. reishi, 100 mg

c. cordyceps, 100 mg,

d. enokitake, 100 mg,

e. chaga, 100 mg

f. maitake, 100 mg

g. mesima, 100 mg

h. turkey tail, 100 mg

i. oyster mushroom, 100 mg

j. agarikon, 100 mg

k. artists conk, 100 mg

l. shiitake, 100 mg

12. xanthan gum, 1.5 g
13. flavored syrup
14. monk fruit 5-10 g
15. water distilled or sourced 12 ounces

makes 12-16 oz

pe 2.9:

### ADD

3 stock 1 top roast 180 mg) 100 mg caff powder
52 ml cold brew 180 mg caffeine 100 mg caff powder->
50 70 90 versions lo med hi onset claims
Use as enhancement of brain meds

### Antidepressants

### Anti-anxiety

Mood disorder

### ADD

### DEMENTIA

Psychotropics—psilocybin

pe 3.0 415 mg caffeine

cold brew conc 135 mg
in 10 oz distilled water
1-2 packet 3^{rd} Wave minerals
3 squirts DreampakTop Roast 180 mg caff
100 mg caffeine powder
Collagen hydrolyzed 8 g+peptides9 g+amino acids (perfotek 0.8 scoop)
MCT oil 0.5 tbsp Nuvina
Lions Mane mush ext liq alc free 20 drops secret of the tribe;
Immune mush ext blend liq alc free 8 drops secret if the tribe;
Lithium orotate 0.35 mg
Vitamin C w rose hips 1000 mg
D-ribose 3.5 g powder
Kate monk fruit sweetener 3 tsp
Jordan skinny cinnamon vanilla 3 tbsp, salted caramel espresso 1 tbsp

### Xanthan gum 1.5 tsp

Mix cycle extract ninja blender
Mix cycle blend ninja blender makes 12-16 oz

pe 3.0 415 mg caffeine

pe 4.0 “high intensity onset”
cold brew conc 135 mg;
in 8 oz distilled water;
0.5 packet 3^{rd} Wave minerals;
3 stock 40 mg caffeine per espresso shots;
90 mg caffeine powder with 180 mg L theanine;
20 mg anhydrous caffeine;
6 drops Horbaach super green tea;
4 drops Hawaii Pham green coffee bean extract;
Collagen hydrolyzed 8 g+peptides9 g+amino acids (perfotek 0.8 scoop);
MCT oil 0.5 tbsp Nuvina;
Lions Mane mush ext liq alc free 20 drops secret of the tribe;
Immune mush ext liq alc free 8 drops secret if the tribe;
Lithium orotate 0.45 mg
Vitamin C w rose hips 1000 mg
D-ribose 3-5 g powder
Kate monk fruit sweetener
Jordan skinny cinnamon vanilla 4 tbsp

### Xanthan gum 0.20 tsp

Mix cycle extract ninja blender
Mix cycle blend ninja blender makes 16 oz

pe 4.1 “focus” (low intensity) onset

cold brew conc 135 mg;
in 8 oz distilled water;
0.5 packet 3^{rd} Wave minerals;
3 stock 40 mg caffeine per espresso shots;
70 mg caffeine powder with 140 mg L theanine;
20 mg anhydrous caffeine;
6 drops Horbaach super green tea;
4 drops Hawaii Pham green coffee bean extract;
Collagen hydrolyzed 8 g+peptides+amino acids (perfotek 0.8 scoop);
MCT oil 0.5 tbsp Nuvina;
Lions Mane mush ext liq alc free 20 drops
secret of the tribe
Immune mush ext liq alc free 8 drops secret if the tribe
Lithium orotate 0.45 mg
Vitamin C w rose hips 1000 mg
D-ribose 3-5 g powder
Kate monk fruit sweetener
Jordan skinny cinnamon vanilla 4 tbsp

### Xanthan gum 0.20 tsp

Mix cycle extract ninja blender
Mix cycle blend ninja blender makes 16 oz

pe 4.1 “focused endorphin” (low intensity) onset

cold brew conc 135 mg;
in 8 oz distilled water;
0.5 packet 3^{rd} Wave minerals;
3 stock 40 mg caffeine per espresso shots;
70 mg caffeine powder with 140 mg L theanine;
20 mg anhydrous caffeine;
10 drops Horbaach super green tea;
4 drops Hawaii Pham green coffee bean extract;
Collagen hydrolyzed 8 g+peptides+amino acids (perfotek 0.8 scoop);
MCT oil 0.5 tbsp Nuvina;
Lions Mane mush ext liq alc free 20 drops secret of the tribe
Immune mush ext liq alc free 8 drops secret if the tribe
Lithium orotate 0.45 mg
Vitamin C w rose hips 1000 mg
D-ribose 3-5 g powder
Kate monk fruit sweetener
Jordan skinny cinnamon vanilla 4 tbsp

### Xanthan gum 0.20 tsp

Mix cycle extract ninja blender
Mix cycle blend ninja blender makes 16 oz

pe 4.2, 4.3 “panic attack”/“sleep” (low intensity) onset

cold brew conc 135 mg;
in 8 oz distilled water;
0.5 packet 3^{rd} Wave minerals;
10 mg caffeine powder with 200 mg L theanine;
14 (panic) drops: 20 (sleep) drops of Horbaach super green tea;
4 drops Hawaii Pham green coffee bean extract;
Collagen hydrolyzed 8 g+peptides+amino acids (perfotek 0.8 scoop);
MCT oil 0.5 tbsp Nuvina;
Lions Mane mush ext liq alc free 27 drops secret of the tribe
Immune mush ext liq alc free 20 drops secret if the tribe
Lithium orotate 0.45 mg
Vitamin C w rose hips 1000 mg
D-ribose 3-5 g powder
Kate monk fruit sweetener
Jordan skinny cinnamon vanilla 4 tbsp

### Xanthan gum 0.20 tsp

Mix cycle extract ninja blender
Mix cycle blend ninja blender makes 16 oz

pe 4.4 “Alzheimer's-Dementia-Cognitive” (low intensity) onset

cold brew conc 135 mg;
in 8 oz distilled water;
0.5 packet 3^{rd} Wave minerals;
3 stock 40 mg caffeine per espresso shots;
60 mg caffeine powder with 120 mg L theanine;
8 drops Horbaach super green tea;
4 drops Hawaii Pham green coffee bean extract;
Collagen hydrolyzed 8 g+peptides+amino acids (perfotek 0.8 scoop);
MCT oil 0.75 tbsp Nuvina;
Lions Mane mush ext liq alc free 28 drops secret of the tribe
Immune mush ext liq alc free 28 drops secret of the tribe;
Lithium orotate 0.65 mg;
Vitamin C w rose hips 2000 mg;
D-ribose 3-5 g powder
Kate monk fruit sweetener
Jordan skinny cinnamon vanilla 4 tbsp

### Xanthan gum 0.20 tsp

Mix cycle extract ninja blender
Mix cycle blend ninja blender makes 16 oz

By further altering caffeine ratios and sources, antioxidants, mushroom blends and lithium micro-dose preferred formulations for depression, suicide ideation, immune boost, glucose level stabilization (diabetics), weight loss, cancer treatment potentiation and side effect toxicity profiles, and sleep-inducing versions can be created to be disclosed individually in formal filing.

TABLE 1
Embodiment X12120

| | Serving | |
|---|---|---|
| | size 12 oz | |
| | Batch | Amount |
| | Size/100,000 | Per |
| | bottles | Bottle |
| Ingredients | grams | grams |
| Vitamin C | 303000 | 3 |
| Citric acid | 40000 | 0.4 |
| Hydrolyzed collagen | 584000 | 5 |
| Hydrolyzed medium | 758000 | 5 |
| chain triglycerides | | |
| D-ribose | 432000 | 4 |
| Erythritol | 303000 | 3.03 |
| Creatine (water stable) | 717000 | 5 |
| Beet root powder | 125000 | 1.25 |
| Green tea extract 4:1 | 25000 | 0.25 |
| GCBE 50% CGA | 75000 | 0.75 |
| L-theanine | 57000 | 0.1 |
| Natural (GCBE) | 35000 | 0.35 |
| caffeine anhydrous | | |
| K Sorbate | 7000 | 0.07 |
| Na Benzoate | 7000 | 0.07 |
| Sucralose | 10000 | 0.1 |
| Vitamin E acetate 50% | 250000 | 2.5 |
| CWS/S | | |
| Zinc citrate dihydrate | 454 | 0.0045 |
| Natural flavor | 39000 | 0.39 |

TABLE 2
Embodiment X22120

| | Serving | |
|---|---|---|
| | size 12 oz | |
| | Batch | Amount |
| | Size/100,000 | Per |
| | bottles | Bottle |
| Ingredients | grams | grams |
| Vitamin C | 100000 | 2 |
| Citric acid | 40000 | 0.4 |
| Hydrolyzed collagen | 584000 | 10 |
| Hydrolyzed medium | 758000 | 12 |
| chain triglycerides | | |
| D-ribose | 432000 | 6 |
| Erythritol | 303000 | 3.03 |
| Creatine (water stable) | 717000 | 7.17 |
| Beet root powder | 125000 | 1.25 |
| Green tea extract 4:1 | 25000 | 0.25 |
| GCBE 50% CGA | 75000 | 0.75 |
| L-theanine | 57000 | 3.5 |
| Natural (GCBE) | 35000 | 0.344 |
| caffeine anhydrous | | |
| K Sorbate | 7000 | 0.07 |
| Na Benzoate | 7000 | 0.07 |
| Sucralose | 10000 | 0.1 |
| Vitamin E acetate 50% | 250000 | 0.5 |
| CWS/S | | |
| Zinc citrate dihydrate | 454 | 0.0025 |
| Natural flavor | 39000 | 0.39 |

Additional embodiments of the present invention include:

A method of enhancing memory, cognitive function, sustained mental focus or a combination thereof comprising administering to a subject in need thereof a composition comprising coffee from roasted coffee beans, and green coffee bean extract.

The method of paragraph 120 wherein the caffeine content from roasted coffee beans is from 0 to 400 mg, more preferably from 30 to 200 mg, and most preferably from 60 to 100 mg.

The method of paragraph 120 wherein chlorogenic acid content from green coffee bean extract is from 15 to 800 mg, more preferably from 50 to 500 mg, and most preferably from 150 to 300 mg.

The method of paragraph 120 wherein the caffeine content of green coffee bean extract is from 0 to 100 mg, more preferably from 20 to 75 mg, and most preferably from 25 to 50 mg.

The method of paragraph 120 wherein the composition further comprises D-ribose at an amount from 0.5 to 10 grams, more preferably from 2 to 8 grams, and most preferably from 4 to 6 grams.

The method of paragraph 120 wherein the composition further comprises medium chain triglyceride (MCT) ketones at an amount from 0.5 and 10 grams, more preferably from 2 to 8 grams, and most preferably from 4 to 6 grams.

The method of paragraph 120 wherein the composition further comprises protein at an amount from 0.5 to 40 grams, more preferably from 5 to 20 grams, and most preferably from 10 to 14 grams.

The method of paragraph 126 wherein the protein is bovine collagen.

The method of paragraph 126 wherein the protein is almond, cashmere, coconut, hemp, pea or other nutmilk extract.

The method of paragraph 126 wherein the protein is vegan or milk protein.

The method of paragraph 126 wherein the protein is one or more of bovine collagen, almond, cashmere, coconut, hemp, pea or other nutmilk extract, vegan or milk protein.

A method of enhancing memory, cognitive function, sustained mental focus or a combination thereof comprising administering to a subject in need thereof a composition comprising coffee from roasted coffee beans, green coffee bean extract, and green matcha tea.

The method of paragraph 131 wherein green matcha tea is at an amount from 0.5 and 6 grams, more preferably from 1 to 5 grams and most preferably from 2 to 4 grams.

The method of paragraph 131 wherein the caffeine content from roasted coffee beans is at an amount from 0 to 400 mg, more preferably from 30 to 200 mg, and most preferably from 60 to 100 mg.

The method of paragraph 131 wherein concentration of chlorogenic acid from green coffee bean extract is from 15 to 800 mg, more preferably from 50 to 500 mg, and most preferably from 150 to 300 mg.

The method of paragraph 131 wherein the caffeine content of green coffee bean extract is from 0 to 100 mg, more preferably from 20 to 75 mg, and most preferably from 25 to 50 mg.

The method of paragraph 131 further comprising D-ribose at an amount from 0.5 to 10 grams, more preferably from 2 to 8 grams, and most preferably from 4 to 6 grams.

The method of paragraph 131 further comprising medium chain triglycerides (MCT) ketones at an amount from 0.5 to 10 grams, more preferably from 2 to 8 grams, and most preferably from 4 to 6 grams.

The method of paragraph 131 further comprising protein at an amount from 0.5 to 40 grams, more preferably from 5 to 20 grams, and most preferably from 10 to 14 grams.

The method of paragraph 138 wherein the protein is bovine collagen.

The method of paragraph 138 wherein the protein is almond, cashmere, coconut, hemp, pea or other nutmilk extract.

The method of paragraph 138 wherein the protein is vegan or milk protein.

The method of paragraph 138 wherein the protein is one or more of bovine collagen, almond, cashmere, coconut, hemp, pea or other nutmilk extract, vegan or milk protein.

The method of paragraphs 120 and 131 wherein the composition further comprises antioxidants.

The method of paragraph 143 wherein the antioxidants is selected form the group consisting of astaxanthin, vitamin C, Vitamin E, cocoa, fruit powder/extract/anthocyanins, resveratrol or a combination thereof

The method of paragraphs 120 and 131 wherein the composition further comprises mushroom extract.

The method of paragraph 145 wherein the mushroom extract is selected from the group consisting of lion's mane, reishi, maitake, enotake, chaga, cordyceps, royal sun blazei, mesoma, turkey tail, oyster mushroom, agarikon, amidou, shiitake or a combination thereof.

The method of paragraph 145 wherein the mushroom extract comprises one or more of polyphenols, polysaccharides, proteases, terpenoids, lectins, illudins, and ribosome inactivating proteins.

The method of paragraphs 120 and 131 wherein the composition further comprises lithium.

The method of paragraph 148 wherein lithium is at an amount from 0.01 mg to 1 mg, more preferably from 0.02 mg to 0.1 mg, and most preferably from 0.025 mg to 0.09 mg.

The method of paragraph 148 where lithium is an orotate, aspartate, chloride, carbonate, or salicylate salt.

The method of paragraphs 120 and 131 further comprising one or more ingredients selected from the group consisting of lithium, antioxidants, and mushroom extract.

A composition for the treatment of carcinomas comprising caffeine, coffee and tea derivatives, medium chain triglycerides, and mushroom extracts.

The composition of paragraph 152 wherein the mushroom extract is lion's mane.

The composition of paragraph 152 wherein the carcinoma is glioblastoma.

The composition of paragraph 152 wherein the carcinoma is hepatic carcinoma.

The composition of paragraph 152 wherein the cancer is leukemia or lymphoma.

The composition of paragraph 152 wherein the cancer is a lung tumor.

The composition of paragraph 152 wherein the cancer is a pancreatic tumor.

The composition of paragraph 152 wherein the cancer is an isrini tumor.

A method of treating a carcinoma comprising administering a composition of paragraph 152.

A composition for the treatment of heart disease containing caffeine, coffee and tea derivatives, medium chain triglycerides, and mushroom extracts.

The composition of paragraph 161 wherein the mushroom extract is lion's mane.

A method of treating heart disease comprising administering a composition of paragraph 161 to a subject in need thereof.

The method of paragraph 163 wherein administration to the subject in need thereof significantly lowers the cholesterol level of the subject.

The method of paragraph 161 wherein administration to the subject in need thereof significantly lowers the low-density lipoproteins of the subject.

The method of paragraph 161 wherein administration to the subject in need thereof significantly increases the high-density lipoproteins of the subject.

The method of paragraph 161 wherein administration to the subject in need thereof reduces the incidence and mortality of myocardial infarction in the subject.

A composition for the prevention or treatment of mental health disorders comprising caffeine, coffee and tea derivatives, medium chain triglycerides, antioxidants, micro-dose lithium, and mushroom extracts.

A method of prevention or treatment of mental disorders comprising administering a composition of paragraph 168 to a subject in need thereof.

The method of paragraph 169 wherein administration to the subject in need thereof reduces the incidence, severity, and or morbidity of cerebral brain hemorrhage in the subject.

The method of paragraph 169 wherein administration to the subject in need thereof reduces mental health morbidity in the subject.

The method of paragraph 169 wherein administration to the subject in need thereof reduces symptoms of post-traumatic stress disorder in the subject.

The method of paragraph 169 wherein administration to the subject in need thereof reduces depression in the subject.

The method of paragraph 169 wherein administration to the subject in need thereof reduces symptoms of bipolar disease in the subject.

The method of paragraph 169 wherein administration to the subject in need thereof decreases the rate of suicide in the subject.

A composition for appetite suppression or weight reduction containing caffeine, coffee and tea derivatives, medium chain triglycerides, protein, antioxidants, micro-dose lithium, and mushroom extracts.

The composition of paragraph 176 further comprising D-ribose.

The composition of paragraph 177 wherein D-ribose is at an amount from 0.5 to 10 g, more preferably from 3 to 8 g, most preferably from 4.5 to 6.5 g.

A composition comprising light medium or dark roasted caffeine, caffeic acid, chlorogenic acid, ferulic acid, green bean extract caffeine, chlorogenic acids, catechins, green tea caffeine, chlorogenic acid, epichallogallatin gallate and D-ribose.

A composition comprising light medium or dark roasted caffeine, caffeic acid, chlorogenic acid, ferulic acid, green bean extract caffeine, chlorogenic acid, catechins, green tea caffeine, chlorogenic acid, epichallogallatin gallate D-ribose and medium chain triglycerides.

A composition comprising light medium or dark roasted caffeine, caffeic acid, chlorogenic acid, ferulic acid, green bean extract caffeine, catechins green tea caffeine, epichallogallatin gallate, lithium, D-ribose medium chain triglycerides and protein.

A composition comprising light medium or dark roasted caffeine, caffeic acid, chlorogenic acid, ferulic acid, green bean extract caffeine, catechins, green tea caffeine, epichallogallatin gallate, lithium, D-ribose, medium chain triglycerides, hydrolyzed collagen, nut milk, and or whey protein.

A composition comprising light medium or dark roasted caffeine, caffeic acid, chlorogenic acid, ferulic acid, green bean extract caffeine, catechins green tea caffeine, epichallogallatin gallate, lithium, D-ribose, medium chain triglycerides, hydrolyzed collagen, nut milk or whey protein, mushroom extract polyphenols, polysaccharides, proteases and terpenoids.

A composition comprising light medium or dark roasted caffeine, caffeic acid, chlorogenic acid, ferulic acid, green bean extract caffeine, catechins, green tea caffeine, epichallogallatin gallate, lithium, D-ribose, medium chain triglycerides, hydrolyzed collagen, nut milk or whey protein, mushroom extract polyphenols, polysaccharides, proteases, terpenoids, and vitamin C.

A composition comprising light medium or dark roasted caffeine, caffeic acid, chlorogenic acid, ferulic acid, green bean extract caffeine, catechins, green tea caffeine, epichallogallatin gallate, lithium, D-ribose, medium chain triglyceride, hydrolyzed collagen, nut milk or whey protein, mushroom extract polyphenols, polysaccharides, proteases, terpenoid, antioxidants, astaxanthin, vitamin C, vitamin E, blueberry anthocyanins, cocoa flavonoids, quercetin, honey and erythritol.

A composition comprising 60 mg of roast coffee bean caffeine and derivatives, caffeine powder, green coffee bean extract, matcha tea powder, D-ribose, medium chain triglycerides as ketones, hydrolyzed collagen and nut milk, pea, or oat protein, mushroom extracts, vitamin C, cocoa, blueberry powder, and astaxanthin, wherein administration of the composition triggers prolonged sustainable mental alertness without inducing hypertension or tachycardia.

A composition comprising 60 mg of roast coffee bean caffeine and derivatives, caffeine powder, green coffee bean extract, matcha tea powder, D-ribose, medium chain triglycerides as ketones, hydrolyzed collagen and nut milk, pea, or oat protein, mushroom extracts, vitamin C, cocoa, blueberry powder, and astaxanthin wherein administration of the composition triggers prolonged sustainable mental alertness without inducing shakiness or jitteriness.

A composition comprising 60 mg of roast coffee bean caffeine and derivatives, caffeine powder, green coffee bean extract, matcha tea powder, D-ribose, medium chain triglycerides as ketones, hydrolyzed collagen and nut milk, pea, or oat protein, mushroom extracts, vitamin C, cocoa, blueberry powder, and astaxanthin wherein administration of the composition triggers prolonged sustainable mental alertness without inducing hyperglycemia.

A composition comprising at least 60 mg of roast coffee bean caffeine and derivatives, caffeine powder, green coffee bean extract, matcha tea powder, D-ribose, medium chain triglycerides as ketones, hydrolyzed collagen and nut milk, pea, or oat protein, mushroom extracts, vitamin C, cocoa, blueberry powder, and astaxanthin wherein administration of the composition triggers prolonged sustainable mental alertness with feelings of well-being.

A composition comprising at least 60 mg of roast coffee bean caffeine and derivatives, caffeine powder, green coffee bean extract, matcha tea powder, D-ribose, medium chain triglycerides as ketones, hydrolyzed collagen and nut milk, pea, or oat protein, mushroom extracts, vitamin C, cocoa, blueberry powder, and astaxanthin wherein administration triggers prolonged sustainable mental alertness with daily or twice daily use.

A composition comprising at least 60 mg of roast coffee bean caffeine and derivatives, caffeine powder, green coffee bean extract, matcha tea powder, D-ribose, medium chain triglycerides as ketones, hydrolyzed collagen and nut milk, pea, or oat protein, mushroom extracts, vitamin C, cocoa, blueberry powder, and astaxanthin wherein administration triggers prolonged sustainable mental alertness and may alleviate depression with regular use.

A composition comprising at least 60 mg of roast coffee bean caffeine and derivatives, caffeine powder, green coffee bean extract, matcha tea powder, D-ribose, medium chain triglycerides as ketones, hydrolyzed collagen and nut milk, pea, or oat protein, mushroom extracts, vitamin C, cocoa, blueberry powder, and astaxanthin wherein administration triggers prolonged sustainable mental alertness and reduces cognitive decline.

A composition comprising at least 60 mg of roast coffee bean caffeine and derivatives, caffeine powder, green coffee bean extract, and or matcha tea powder; two or more of D-ribose, medium chain triglycerides as ketones, hydrolyzed collagen, and whey, nut milk, pea, oat or other protein; mushroom extract; one or more of vitamin C, cocoa, blueberry powder, and astaxanthin; wherein administration triggers prolonged sustainable mental alertness and with regular administration reduces the degree of cognitive impairment in Alzheimer's disease or other forms of dementia.

A composition comprising at least 60 mg of roast coffee bean caffeine and derivatives and or caffeine powder; one or more of green coffee bean extract, matcha tea powder; on or more of D-ribose, medium chain triglycerides as ketones, hydrolyzed collagen and or nut milk, pea, or oat protein; mushroom extracts including lions mane; antioxidants including one or more of vitamin C, cocoa, blueberry powder, olive oil or extract, and astaxanthin; wherein administration of the composition triggers prolonged sustainable mental alertness, and with regular use reduces the degree of cognitive impairment in traumatic brain injury.

A composition comprising at least 100 mg of roast coffee bean caffeine and derivatives and or caffeine powder, green coffee bean extract, matcha tea powder, D-ribose, medium chain triglycerides as ketones, hydrolyzed collagen, vitamin C, and or cocoa, blueberry powder, and astaxanthin; wherein administration of the composition triggers prolonged sustainable mental alertness and with regular use reduces the frequency of migraine headaches.

A composition comprising at least 60 mg of roast coffee bean caffeine and derivatives, caffeine powder, green coffee bean extract, matcha tea powder, D-ribose, medium chain triglycerides as ketones, hydrolyzed collagen and nut milk, pea, or oat protein, mushroom extracts, vitamin C, cocoa, blueberry powder, and astaxanthin wherein administration of the composition triggers prolonged sustainable mental alertness and with regular use improves short term memory retention.

A composition comprising from 60 mg to 350 mg of caffeine sourced from roast coffee bean coffee and derivatives and or caffeine powder, green coffee bean extract, matcha tea powder; metabolic fuel sourced from one or more of D-ribose, medium chain triglycerides, hydrolyzed collagen, collagen peptides and or nut milk, pea, whey, oat milk, or vegan protein; micro dose lithium; a mushroom extract blend including lions mane, reishi, enotake, maitake, and chaga; added antioxidants with one or more of vitamin C, cocoa, blueberry powder, resveratrol and or astaxanthin; wherein administration of the composition triggers prolonged sustainable mental alertness without significant side effects, is anxiolytic, induces sleep transition and quality, improves mental focus and memory retention, and induces feelings of wellbeing.

A composition of PE 3.0 of paragraph 111 or 112 in the specification wherein administration of the composition causes 12 hours of sustained alertness is realized, with ease of sleep transition and endorphin like wellbeing on arising.

A composition of PE 3.0 of paragraph 111 or 112 in the specification wherein administration of the composition causes the onset of alertness can be managed by increasing caffeine powder or decreasing green tea and vice versa.

A composition to induce enhanced brain function and brain and systemic recovery comprising:

a. a brain stimulant derived from a source selected from the group consisting of caffeine powder, cold or hot brewed caffeinated roast coffee, a non-caffeine stimulant and a combination thereof;
b. one or more beverages selected from the group consisting of unroasted green coffee bean extract, black tea, white tea, green tea, green tea concentrate, and matcha tea; and
c. an energy source selected from the group consisting of a low glycemic carbohydrate, preferably the low glycemic carbohydrate is selected from the group consisting of D-ribose, fructose, lactose and sucrose, a medium chain saturated triglyceride, preferably selected from the group consisting of C8, C10, and C12 fatty acids, a source of amino acids, preferably the source of amino acids is selected from the group consisting of protein, peptides, collagen, hydrolyzed collagen, and twice hydrolyzed collagen.

A method of enhancing brain function and brain and systemic recovery comprising administering to a subject in need thereof a composition of paragraph 200.

The method of paragraph 120 wherein the composition comprises from 100 to 120 mg caffeine anhydrous and further comprises from 160 to 200 mg L theanine.

The method of paragraph 120 wherein the composition comprises from 80 mg caffeine anhydrous and further comprises from 120 to 140 mg L theanine.

A method of enhancing antimicrobial and antiviral potency of a food, supplement, or beverage, wherein the composition includes zinc and one or more of green coffee bean extract, green tea extract, or polyphenols.

The method of paragraph 204 wherein the cumulative amount of green coffee bean and green tea extract is at least 0.50 mg, preferably 300 mg, and most preferably 720 mg.

The method of paragraph 204 wherein the ratio of green coffee bean to green tea is at least 2:1, preferably 3:1, and most preferably 4:1 or greater.

A preferred embodiment, X22120, wherein the ratio of L-theanine to caffeine is at least 0.25:1, more preferably 0.75:1, and most preferably 1.65:1.

A preferred embodiment X22120, wherein vitamin C oxidation is suppressed by encapsulating vitamin C in an insoluble matrix, wherein the particle size is less than 200 microns.

The method of any of the preceding paragraphs in which shots of 4 oz of X022120 contain 50-200 mg of caffeine.

The method of paragraph 209 wherein an embodiment contains 0-100 mg of caffeine for purposes of reduction of oxidative stress.

The method of paragraph 209 which result in appetite suppression.

The method of paragraph 209 in which an embodiment is used to break fasts in intermittent fasting.

The method of paragraph 209 in which feelings of wellness are increased with regular use.

### EXAMPLES

The following examples demonstrate profound recovery lack of sleep and profound recovery and include prophetic examples caffeine absorption multi options, prophetic examples fuels, prophetic examines dual absorption multi options for results, prophetic examples both+antioxidants and prophetic examples mushroom extracts.

Examples 1-4 assess heart rate, alertness, and anxiety—relaxation.

Heart rate, avg: radial pulse 1 min×2
Alertness subjective 1-10 scale 100 mg cup caffeinated coffee:

| | |
|---|---|
| 0.0 | asleep |
| 1.0 | groggy |
| 2.5 | ½ cup |
| 5.0 | 1 cup |
| 7.5 | 5 1.5 cups |
| 10.0 | 2 cups |

### Example 1 21-Year-Old is Tested for Alertness, Relaxation, and Heart Rate after Consuming BW.pe 2.1

Consumed beverage over a 30-minute time frame. No health issues.

Heart rate:

| | |
|---|---|
| =baseline | 66 |
| =two hours post | 60 |

Alertness

| | |
|---|---|
| =baseline describes sense of fatigue | 1 |
| =30 minutes post | 3 |
| =1 hr post | 8 |
| =4 hours post | 4 |

Relaxation

| | |
|---|---|
| =baseline | 0 |
| =30 minutes post | not assessed |
| =1 hour post | 4 |
| =4 hours post | 8 |
| =6 hours post | |
| =sleep | |
| =next day on arising | |
| Side effects, other: none | |

### Example 2 67-Year-Old is Tested for Alertness, Relaxation, and Heart Rate after Consuming BW.pe 2.2

Heart rate:

| | |
|---|---|
| =baseline | 74 |
| =two hours post | 66 |

Alertness

| | |
|---|---|
| =baseline describes neutral | 2 |
| =30 minutes post | 3 |
| =1 hr post | 6 |
| =4 hours post | 4 |

Relaxation

| | |
|---|---|
| =baseline | 0 |
| =30 minutes post | not assessed |
| =1 hour post | 3 |
| =4 hours post | 6 |
| =6 hours post | 6 |
| =sleep | 8 |
| =next day AM, arising | 10 |
| Side effects, other: none | |

### Example 3 67-Year-Old is Tested for Alertness, Relaxation, and Heart Rate after Consuming BW.pe 2.3

Heart rate:

| | |
|---|---|
| =baseline | 70 |
| =15 min post | 68 |
| =30 min post | 67 |
| =1 hr post | 68 |
| =2 hrs post | 65 |
| =4 hrs post | 71 |
| =6 hrs post | 70 |

Alertness

=baseline describes neutral
=30 minutes post
=1 hr post
=4 hours post

Relaxation

| | |
|---|---|
| =baseline | |
| =30 minutes post | not assessed |
| =1 hour post | |
| =4 hours post | |
| =6 hours post | |
| =sleep | |
| =next day AM, arising | |
| Side effects, other: none | |

### Example 4 67-Year-Old is Tested for Alertness, Relaxation, and Heart Rate after Consuming BW.pe 2.5

Heart rate:

| | |
|---|---|
| =baseline | 81 |
| =15 min post | |
| =30 min post | |
| =1 hr post | |
| =2 hrs post | |
| =4 hrs post | |
| =6 hrs post | |

Alertness

=baseline describes neutral
=30 minutes post
=1 hr post
=4 hours post

Relaxation

| | |
|---|---|
| =baseline | |
| =30 minutes post | not assessed |
| =1 hour post | |
| =4 hours post | |
| =6 hours post | |
| =sleep | |
| =next day AM, arising | |
| Side effects, other: none | |

Examples 6-8 are prophetic examples of serum levels versus time of one or more of caffeine, ribose, amino acids, ketones, vitamin C, astaxanthin, resveratrol, anthocyanins, lithium, and mushroom extract derivatives (on or more of polyphenols, polysaccharides, proteases, terpenoids, lectins, illudins, ribosome inactivating proteins).

### Example 6

Blood glucose, ketone levels, and serum caffeine levels were prophetically measured at baseline, 1 hour, 4 hours, 8 hours and on arising. Heart rate and blood pressure were also measured.

D-ribose serum levels, amino acid levels, ketone levels and caffeine were compared from baseline to hour 8. Comparison was made to black coffee, GCBE, and matcha tea.

### Example 7

Pe 3.0 was compared with 415 mg 1 ml super green tea 0.5 ml. No noticeable onset at 1 ml though at 1-hour considerable added alertness imperceptibly increased. This was compared to 0.5 ml and alertness increase with slight jitteriness manifested for about 5 min. 12 hours total alertness which was super acute at least 8 hours.

### Example 8

Label: drink 1 16 oz serving for reduced mental fatigue and enhanced mental alertness.

Serving size recommended: 1 per day. Do not exceeds 2 servings per day.

TABLE 3

| | | | | | | | | | | | Fat, | Fat, | |
|---|---|---|---|---|---|---|---|---|---|---|---|---|---|
| | | | | | | | [symbol] | CGA's | [symbol] | Fat T | sat | tran [symbol] | Chol |
| Excipient | Dose | | Active n | | Cal | Caff | mg | mg | mg | g | g | g | mg |
| Death Wish Coffee, cold brew | 8 | oz | | | 15.00 | 300 | | | | | | | |
| Hawaii Pharm Green Coffee | 1.5 | ml | 500 | mg | | 15 | | 25 [symbol] | | | | | |
| 1 ml = 970 mg −> 330 mg GCBE | | | | | | | | | | | | | |
| Superfoods Green Matcha tea | 2 | tsp | 4 | g | | 64 | 91.3 | | 248.9 | 9 | 9 | 9 | 9 |
| Health Direct Collagen Amino Sculpt | 0.5 | tbsp | | | 45.00 | | | | | [symbol] | 0 | 0 | 0 |
| Nutiva MCT Oil | 0.5 | tbsp | 15 | ml | 65.00 | | | | | 7 | 7 | 0 | 0 |
| Bulksupplements D-ribose | 0.5 | tbsp | 5.53 [symbol] | g | 18.00 | | | | | | | | |
| Barleans Olive Leaf Complex | 0.5 | tbsp | 15 | ml | 22.50 | | | | | | | | |
| Dynamic Health Liq Vit C | 0.25 | tbs [symbol] | 250 | mg | 5.00 | | | | | | | | |
| Nature's Answer Resvetrol Complex | 0.25 | tsp | | | 3.75 | | | | | | | | |
| Life Cykel Lions mane Kakadu plum | 1 | ml | 280. [symbol] | mg | | | | | | | | | |
| blend 1:3 extraction | | | | | | | | | | | | | |
| Maui Herbs LLC | 1 | ml | 682 | mg | | | | | | | | | |
| KAL Lithium Orotate | 0.40 | mg | 400 | mc [symbol] | | | | | | | | | |
| Mineral TDS 50-150; 2:1 ratio | | | | | | | | | | | | | |
| (ca + mg):hco3, max hco3 75 ppm | | | | | | | | | | | | | |
| Distilled water | | | | | | | | | | | | | |
| TOTAL | | | | | | | | | | | | | |

| | Protein | MCT Oil | C8 | C10 | C12 | T Carbs | Sugars | DtaryFb | Mg | Na | K |
|---|---|---|---|---|---|---|---|---|---|---|---|
| Excipient | g | g | g | g | g | g | g | g | mg | mg | mg |
| Death Wish Coffee, cold brew | 1 | | | | | 4 | | | | 60 | 166 |
| Hawaii Pharm Green Coffee | | | | | | | | | | | |
| 1 ml = 970 mg −> 330 mg GCBE | | | | | | | | | | | |
| Superfoods Green Matcha tea | 1 | 0 | | | | 2 | 0 | 1 | | | 40 |
| Health Direct Collagen Amino Sculpt | 8 | | | | | 3 | 0 | | | | |
| Nutiva MCT Oil | | 6.5 | 3.65 | 2.5 | 0.35 | | | | | | |
| Bulksupplements D-ribose | | | | | | 4.5 | 4.5 | | | | |
| Barleans Olive Leaf Complex | | | | | | 5 | | | | | |
| Dynamic Health Liq Vit C | | | | | | 1.5 | 0.5 | | | | |
| Nature's Answer Resvetrol Complex | | | | | | | | | | | |
| Life Cykel Lions mane Kakadu plum | | | | | | | | | | | |
| blend 1:3 extraction | | | | | | | | | | | |
| Maui Herbs LLC | | | | | | | | | | | |
| KAL Lithium Orotate | | | | | | | | | | | |
| Mineral TDS 50-150; 2:1 ratio | | | | | | | | | | | |
| (ca + mg):hco3, max hco3 75 ppm | | | | | | | | | | | |
| Distilled water | | | | | | | | | | | |
| TOTAL | | | | | | | | | | | |

| | | | | | | Vit C | | Resvet |
|---|---|---|---|---|---|---|---|---|
| Excipient | Cl | Ca | Iron | Mg | Cl | mg | Vit A | mg |
| Death Wish Coffee, cold brew | | | | | | | | |
| Hawaii Pharm Green Coffee | | | | | | | | |
| 1 ml = 970 mg −> 330 mg GCBE | | | | | | | | |
| Superfoods Green Matcha tea | | 2% | 4% | | | 16% | 10% | |
| Health Direct Collagen Amino Sculpt | | | | | | | | |
| Nutiva MCT Oil | | | | | | | | |
| Bulksupplements D-ribose | | | | | | | | |
| Barleans Olive Leaf Complex | | | | | | | | |
| Dynamic Health Liq Vit C | | | | | | 250 | | |
| Nature's Answer Resvetrol Complex | | | | | | 30 | | 62.5 |
| Life Cykel Lions mane Kakadu plum | | | | | | | | |
| blend 1:3 extraction | | | | | | | | |
| Maui Herbs LLC | | | | | | | | |
| KAL Lithium Orotate | | | | | | | | |
| Mineral TDS 50-150; 2:1 ratio | | | | | | | | |
| (ca + mg):hco3, max hco3 75 ppm | | | | | | | | |
| Distilled water | | | | | | | | |
| TOTAL | | | | | | | | |

5-6 ounce 6 samples unflavored
5-6 ounce 6 samples flavored
Flavoring Goals: 1&2 both key
1 mask bitterness
2 add back coffee flavor lost
3 slightly sweet
4 optional: mocha, caramel, vanilla creme, combinations [symbol]
indicates data missing or illegible when filed

### Example 9 (Prophetic)

100 subjects were prophetically divided into controls vs Breinfuel™ X12120. The following 7 cognitive variables were compared: finger tapping (speed); verbal memory; visual memory; symbol digit coding; Stroop test; shifting attention test; digit span test; continuous performance test.

Breinfuel™ demonstrated statistically significantly cognitive improvement in each of the variables studied.

### Example 10 (Prophetic)

A two yearlong prophetic study on subjects with early dementia was conducted with controls on Breinfuel™ X12120 but altered to 180 mg caffeine. After completing the study, a statistically significant reduction in cognitive decline was noted in the Breinfuel™ cohort.

### Example 11 (Prophetic)

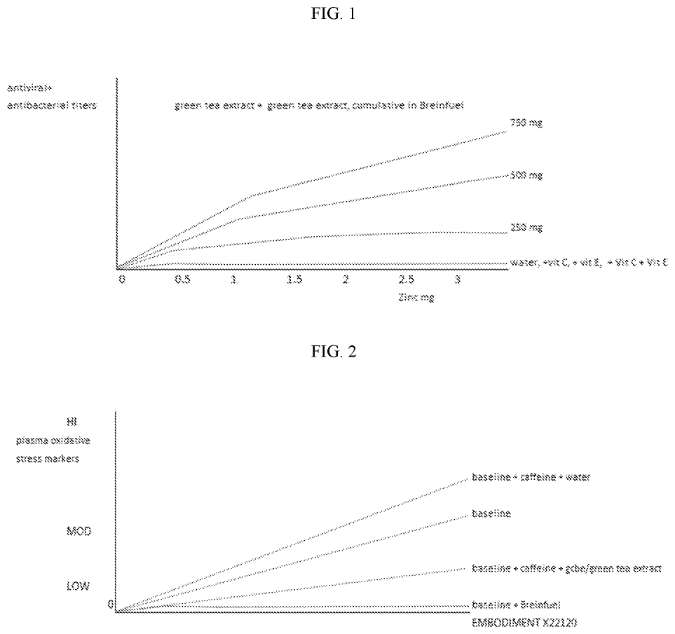

The addition of zinc at 250, 500, and 750 mg to Breinfuel™ resulted in increased antiviral and antibacterial titers. See FIG. 1.

### Example 12 (Prophetic)

The addition of GCBE and green tea extract to Breinfuel™ resulted in decreased levels of plasma oxidative stress markers. See FIG. 2.

What is claimed is:

1. A composition comprising caffeine, coffee and tea derivatives, medium chain triglycerides, and mushroom extracts.

2. The composition of claim 1 wherein the mushroom extract is lion's mane.

3. A method of treating cancer comprising administering a composition of claim 11 to a subject in need thereof.

4. The method of claim 3, wherein the cancer is selected from the group consisting of glioblastoma, carcinoma, leukemia and lymphoma.

5. The method of claim 3, wherein the cancer is selected from the group consisting of a lung tumor, a pancreatic tumor and an isrini tumor.

6. A method of treating heart disease heart disease administering a composition of claim 1 to a subject in need thereof.

7. The method of claim 6 wherein administration further causes a reaction selected from the group consisting of lowering the cholesterol level, lowering low-density lipoproteins, increasing high-density lipoproteins, reducing the incidence and mortality of myocardial infarction and combinations thereof.

8. A composition comprising caffeine, coffee and tea derivatives, medium chain triglycerides, antioxidants, micro-dose lithium, and mushroom extracts.

9. A method of prevention or treatment of mental disorders comprising administering a composition of claim 8 to a subject in need thereof.

10. The method of claim 9 wherein administration to the subject in need thereof causes a reaction selected from the group consisting of reducing the incidence, severity, and or morbidity of cerebral brain hemorrhage, reducing mental health morbidity, reducing symptoms of post-traumatic stress disorder, reducing depression, reducing symptoms of bipolar disease, decreasing likelihood of suicide and combinations thereof.

11. The composition of claim 8 further comprising protein.

12. The composition of claim 11 further comprising D-ribose.

13. A composition to induce enhanced brain function and brain and systemic recovery comprising:
a. a brain stimulant derived from a source selected from the group consisting of caffeine powder, cold or hot brewed caffeinated roast coffee, a non-caffeine stimulant and a combination thereof;
b. one or more beverages selected from the group consisting of unroasted green coffee bean extract, black tea, white tea, green tea, green tea concentrate, and matcha tea; and
c. an energy source selected from the group consisting of a low glycemic carbohydrate, preferably the low glycemic carbohydrate is selected from the group consisting of D-ribose, fructose, lactose and sucrose, a medium chain saturated triglyceride, preferably selected from the group consisting of C8, C10, and C12 fatty acids, a source of amino acids, preferably the source of amino acids is selected from the group consisting of protein, peptides, collagen, hydrolyzed collagen, and twice hydrolyzed collagen.

14. A method of enhancing brain function and brain and systemic recovery comprising administering to a subject in need thereof a composition of claim 13.

15. The method of claim 14 wherein the composition comprises from 100 to 120 mg caffeine anhydrous and further comprises from 160 to 200 mg L theanine.

16. The method of claim 14 wherein the composition comprises from 80 mg caffeine anhydrous and further comprises from 120 to 140 mg L theanine.
